(19) United States
(12) Patent Application Publication
(10) Pub. No.: US 2008/0021205 A1
(43) Pub. Date: Jan. 24, 2008

(54) Methods and Compositions for Use in Preparing Hairpin Rnas

(76) Inventors: Helen Blau, Menlo Park, CA (US); George Sen, Stanford, CA (US); Jason Meyers, Menlo Park, CA (US); Tom Wehrman, Fremont, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 10/581,503

(22) PCT Filed: Dec. 10, 2004
(86) PCT No.: PCT/US04/41569

§ 371 Date: Jun. 28, 2007

Related U.S. Application Data
(60) Provisional application No. 60/529,407, filed on Dec. 11, 2003.
(60) Provisional application No. 60/532,506, filed on Dec. 26, 2003.

(51) Int. Cl. C07H 19/00; C07H 21/02

(52) U.S. Cl. 536/23.1; 435/183; 536/22.1; 536/25.3

(57) ABSTRACT

Methods and compositions for producing hRNA, e.g., shRNA, expression modules for specific target nucleic acids are provided. In the subject methods, an initial nucleic acid, e.g., dsDNA, synthetic DNA, etc., corresponding to the target nucleic acid of interest is converted to an intermediate nucleic acid. The resultant intermediate nucleic acid, following an optional size modification step, is then converted to a linear dsDNA that includes at least one copy of the hRNA expression module of interest, or a precursor (i.e., pro-shRNA expression module) thereof, where in certain embodiments conversion may include amplification. Also provided are reagents, systems and kits for use in practicing the subject methods. The subject methods and compositions find use in a variety of different applications.

### CROSS-REFERENCE TO RELATED APPLICATIONS

Pursuant to 35 U.S.C. § 119 (e), this application claims priority to the filing date of the U.S. Provisional Patent Application Ser. No. 60/532,506 filed Dec. 26, 2003 and U.S. Provisional Patent Application Ser. No. 60/529,407 filed Dec. 11, 2003; the disclosures of which are herein incorporated by reference.

### GOVERNMENT RIGHTS

This invention was made with government support under federal grant nos. GM08412; AG00259; AG09521; AG20961; HL65572; and HD18179 awarded by the National Institutes of Health. The United States Government may have certain rights in this invention.

### INTRODUCTION

### Background of the Invention

The advent of RNA interference (RNAi) technology has provided a rapid means for assessing the loss of function effects of any gene in the genome. RNAi specifically reduces a single mRNA species by the introduction of its corresponding double-stranded RNA (dsRNA).

Initially, the technology was limited to Drosophila and C. Elegans, because long dsRNA induces an interferon response in most mammalian cell types and a subsequent non-specific inhibition of mRNA translation. In Drosophila, long dsRNA was shown to be cleaved to produce small 21-23 nucleotide (nt) dsRNA (siRNA) molecules that were the effectors of gene silencing.

It was subsequently demonstrated in mammalian cells that transfection of these small dsRNA molecules could circumvent the interferon response and efficiently target specific mRNAs for elimination. However, this effect was transient due to loss of the transfected siRNA by degradation or dilution via cell division.

To overcome this limitation, plasmid vectors were designed to encode short hairpin RNAs (i.e., short hairpin RNA molecules, shRNAs) with structures similar to active siRNA molecules. The continual production of these transcripts allowed long term silencing of genes via siRNA. The plasmid based RNAi systems provided a flexible platform for siRNA production that led to the development of several vector types, transfection based, retroviral, lentiviral, and regulatable systems.

Despite these remarkable advances, several factors currently limit the use of plasmid-based siRNAs in mammalian cells. DNA encoded siRNAs are sequence-specific and have a palindromic hairpin structure. As a result, siRNA vectors for a given gene must be constructed individually using sequence specific oligonucleotide primer pairs. Because only 25% of selected sequences are functional, for reasons that have yet to be identified, a minimum of four constructs must be synthesized and cloned for each gene. Although feasible for one or a few genes, targeting every gene in the human genome would require approximately 160,000 individual constructs.

As such, there is significant interest in the development of new ways to produce siRNA encoding plasmids, where of particular interest would be the development of a protocol that overcomes one or more of the disadvantages experienced with the currently employed protocols.

### Relevant Literature

Of interest are U.S. Pat. Nos.; 6,506,559; and 6,573,099. Also of interest are the following published patent applications: US—2002/00863561A1; US—2003/0108923 A2; WO 99/32619; WO 99/49029; WO 01/36646A1; WO 01/68836A2; WO 01/70949A1; WO 02/44321A2; WO 02/055693A2; DE 199 56 568A1; DE 101 00 586C1 and DE 101 00 588 A1. Journal articles of interest include: Bass et al., Cell (2000) Vol. 101:235-238; Bernstein et al., RNA (2001) 7: 1509-1521; Bernstein et al., Nature (2001) 409:363-366; Billy et al., Proc. Nat'l Acad. Sci USA (2001) 98:14428-33; Caplan et al., Proc. Nat'l Acad. Sci USA (2001) 98:9742-7; Carthew et al., Curr. Opin. Cell Biol (2001)13: 244-8; Clemens et al. Proc. Nat'l Acad. Sci. USA (2000) Vol. 97: 6499-6503; Elbashir et al., Nature (2001) 411: 494498; Gitlin et al., Nature (2002) 418:430-434; Hammond et al., Science (2001) 293:1146-50; Hammond et al., Nat. Ref. Genet. (2001) 2:110-119; Hammond et al., Nature (2000) 404:293-296; Kennerdel et al., Nat. Biotechnology (2000) Vol.17: 896-898; McCaffrrey et al., Nature (2002): 418-38-39; McCaffrey et al., Mol. Ther. (2002) 5:676-684; Paddison et al., Genes Dev. (2002) 16:948-958; Paddison et al., Proc. Nat'l Acad. Sci USA (2002) 99:1443-48; Smalheiser et al., Trends Neurosciences (2001) Vol. 24: 216-218; Sui et al., Proc. Nat'l Acad. Sci USA (2002) 99:5515-20; and Yang et al., Proc. Nat'l Acad. Sci USA (2002) 99: 9942-9947.

### SUMMARY OF THE INVENTION

Methods and compositions for producing hairpin RNA expression modules, e.g., shRNA expression modules, for specific target nucleic acids are provided. In the subject methods, an initial nucleic acid, e.g., dsDNA, synthetic DNA, etc., corresponding to the target nucleic acid of interest is converted to an intermediate nucleic acid. The resultant intermediate nucleic acid is then converted to a linear dsDNA that includes at least one copy of the shRNA expression module of interest, or a precursor (i.e., pro-shRNA expression module) thereof. Also provided are reagents, systems and kits for use in practicing the subject methods. The subject methods and compositions find use in a variety of different applications, including the production of shRNA molecules specific for target genes, and the production of libraries of shRNA molecules.

### BRIEF DESCRIPTION OF THE FIGURES

FIG. 1 provides a schematic view of a representative embodiment of the subject methods. (Step 1) The genes to be silenced are first fragmented using diverse restriction enzymes, Hinpl, BsaHl, Acil, Hpall, HypCHIV, and Taq∝l that exist with high frequency in the genome and result in the same 2 nucleotide overhang to facilitate cloning (CG). The basis for this step is ultimately to generate as many siRNA constructs per gene as possible. (Step 2) These fragments are ligated to a linker oligonucleotide, that forms a hairpin loop (3′ loop), to link the sense and antisense strands. The 3′ loop was engineered to contain a sufficiently long double-stranded stretch to allow efficient self-annealing and ligation by T4 DNA ligase. Since the 3′ loop sequence had to be longer than that accommodated in a non-interferon inducing transcribed siRNA, a BamHl restriction enzyme site was engineered into the 3′ loop to eliminate this extraneous sequence after the first cloning reaction (see step 6 below). To limit the size of the gene-specific fragments that would be transcribed into siRNAs, a recognition sequence for the Mmel restriction enzyme which cleaves exactly 20 base pairs from its recognition site, was engineered into the 3′ loop. Thus, upon cleavage with this enzyme all fragments that were ligated to the 3′loop are now of functional size. (Step 3) A second linker nucleic acid, noted in the Figure as a 5′ hairpin loop, was engineered to contain two specific restriction sites essential to subsequent cloning into the expression vector. Ligation of the 5′loop to the Mmel digested product resulted in the generation of a single-stranded closed circular dumbbell structure. (Step 4) Rolling circle amplification is used to amplify the product of the second ligation reaction and to create linear double stranded DNA for cloning. The DNA polymerase used in RCA causes displacement of the newly synthesized strand, allowing repeated replication. As a result, RCA of the ligation product yields a concatemer of palindromic double-stranded DNA encoding siRNA molecules. (Step 5) Digestion with Bglll and Mlyl allows insertion into vREGS. (Step 6) The plasmids are digested with BamHl to eliminate the extraneous sequence, and then religated forming the final product: expression-ready siRNA vectors. The transcribed product is shown at the bottom as a product of REGS in comparison with those obtained from conventional cloning into pSuper.

FIG. 2 shows generation of multiple siRNA constructs using the REGS process exemplified in FIG. 1. (a) Ligation of the 3′ loop to restriction enzyme digested glucocorticoid receptor(GR) followed by Mmel digestion. Lane 7 shows the glucocorticoid receptor(GR) digested with the restriction enzymes, Hinpl, BsaHl, Acil, Hpall, HypCHIV, and Taq∝l. The digested GR fragments were ligated to the 3′ loop as seen by the upward shift in bands in lane 5. Ligation of the 3′loop to GR fragments followed by digestion with Mmel results in the appearance of a band at 34 bp which corresponds to the 3′loop+21 bp of GR sequence (lane 6). The predominant band at approximately 30 bp in lanes 4-6 is the 3′loop self-ligated. (b) Ligation of the 5′ loop to GR fragments-3′loop. The 5′loop was self-ligated forming a 45 bp band as shown in lane 3. Lane 4 shows ligation of the 5′ loop to GR fragments-3′loop resulting in the desired 60 bp product. (c) Generation of palindromic double stranded DNA encoding siRNA molecules. RCA using primers towards the 5′loop was performed on all samples. Digestion with Bglll/Mlyl of the 5′loop-GR fragments-3′loop shows the appearance of the expected 82 bp band (black arrowhead) containing the desired product and a 38 bp band containing the remnants of the 5′ loop (lane 7). Lane 3 shows that digestion with Bglll/Mlyl of the self-ligated 5′loop results in the expected 38 bp band. Partially digested fragments are indicated by the white arrows in lanes 3 and 7 that appear with varying intensities from experiment to experiment.

FIG. 3 shows the generation of multiple GFP siRNA constructs and the knockdown of GFP expression. (a) Flow cytometry analysis of siRNA constructs targeting GFP. Primary myoblasts constitutively expressing GFP were transduced with siRNA constructs targeting GFP. VREGS was used as a negative control and the parental myoblasts show the autofluorescent baseline value. The upper panel compares the silencing efficiency between the same siRNA sequence targeting GFP cloned using the pSuper loop (pSuper 489) or the vREGS loop (REGS GFP 489). The bottom panel shows four REGS constructs that knockdown GFP expression to varying degrees. (b) Western blot analysis of GFP siRNA constructs. VREGS and an siRNA construct targeting the Oct-3/4 gene, REGS Oct-792, were used as negative controls (lanes 1 and 2). pSuper 489 and REGS GFP 489 show similar knockdowns indicating the vREGS loop does not adversely affect gene silencing. The four REGS constructs derived from the REGS procedure that successfully silenced GFP by flow cytometry also show knockdown by Western blot (lanes 5-8). Percent GFP knockdown was calculated by normalizing to the loading control, α-tubulin. (c) GFP digested with restriction enzymes Hinpl, BsaHl, Acil, Hpall, HpyCHIV, and Taq∝l. The sequences of siRNA constructs isolated from GFP are shown in red. Cyan indicates the constructs that were possible but not isolated. Regions in green are sequences too far away from a restriction site or too short to be functional as an siRNA. The numbered bars below the diagram show the extent of each siRNA that could be isolated, and corresponds to the numbered sequences in d. (d) Frequency of each siRNA construct towards different regions of GFP isolated. 26 siRNA constructs against GFP can be generated. 18 of the possible 26 constructs were isolated, 9 antisense and 9 sense. The asterisk denotes sequences that were able to silence GFP expression.

FIG. 4 shows the generation of multiple siRNA constructs and silencing of Oct-3/4 expression. (a) Semi-quantitative RT-PCR analysis of Oct-3/4 expression. siRNA constructs targeting Oct-3/4 were transduced into ES cells. Three REGS derived constructs showed silencing of Oct-3/4 expression by semi-quantitative PCR (lanes 4-6). pSuper Oct 792 was used as a positive control. vREGS and REGS GFP 10 were used as negative controls. (b) Knockdown of Oct-3/4 results in loss of alkaline phosphatase expression and differentiation of embryonic stem cells into trophoblasts. REGS Oct 58, 522, and 782 transduced cells that showed knockdown by RT-PCR (a) differentiated into trophoblasts as shown by a large flattened morphology and loss of alkaline phosphatase expression. Cells transduced with an irrelevant siRNA (REGS GFP 10) showed no trophoblast formation. (c) Knockdown of Oct-3/4 expression causes downregulation of ES cell specific genes, ESG1 and UTF1 while upregulating H19, a gene associated with differentiation by semi-quantitative PCR.

FIG. 5 shows the knockdown of MyoD expression. (a) Silencing of MyoD expression blocks terminal differentiation of myoblasts. Primary myoblasts constitutively expressing GFP were transduced with REGS construct MyoD 620 or the negative control vREGS and cultured in differentiation medium (5% horse serum) for 2 days. REGS MyoD 620 completely prevented differentiation of myoblasts to myotubes. Cells were also stained for α-sarcomeric actin, a cytoskeletal protein found only in differentiated myotubes. (b) Western blot analysis of MyoD knockdown using siRNA construct REGS MyoD 620. Primary myoblasts constitutively expressing GFP were transduced with various siRNA constructs targeting MyoD. Total protein was isolated and Western blot analysis shows a 10-fold reduction in the levels of MyoD by REGS MyoD 620.

FIG. 6 shows sequences isolated from the REGS siRNA library. 50 clones from the original library were isolated and sequenced. The position of the gene that matches the coding siRNA is indicated in the center. The symbol on the left indicates the orientation of the sequence in the vector (+sense, −antisense). Of the 50 sequences 48 contained the proper sized inserts, 3 inserts were from contaminating vector sequences, and 3 had no identical matches in the Genbank database. 20 were cloned in the sense orientation and 22 were antisense. All sequences isolated were unique.

### DEFINITIONS

For convenience, certain terms employed in the specification, examples, and appended claims are collected here.

As used herein, the term “vector” refers to a nucleic acid molecule capable of transporting another nucleic acid to which it has been linked. One type of vector is a genomic integrated vector, or “integrated vector”, which can become integrated into the chromosomal DNA of the host cell. Another type of vector is an epifocal vector, i.e., a nucleic acid capable of extra-chromosomal replication. Vectors capable of directing the expression of genes to which they are operatively linked are referred to herein as “expression vectors”. In the present specification, “plasmid” and “vector” are used interchangeably unless otherwise clear from the context.

As used herein, the term “nucleic acid” refers to polynucleotides such as deoxyribonucleic acid (DNA), and, where appropriate, ribonucleic acid (RNA). The term should also be understood to include, as applicable to the embodiment being described, single-stranded (such as sense or antisense) and double-stranded polynucleotides.

As used herein, the term “gene” or “recombinant gene” refers to a nucleic acid comprising an open reading frame encoding a polypeptide of the present invention, including both exon and (optionally) intron sequences. A “recombinant gene” refers to nucleic acid encoding such regulatory polypeptides, that may optionally include intron sequences that are derived from chromosomal DNA. The term “intron” refers to a DNA sequence present in a given gene that is not translated into protein and is generally found between exons. As used herein, the term “transfection” means the introduction of a nucleic acid, e.g., an expression vector, into a recipient cell by nucleic acid-mediated gene transfer.

A “protein coding sequence” or a sequence that “encodes” a particular polypeptide or peptide, is a nucleic acid sequence that is transcribed (in the case of DNA) and is translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, cDNA from procaryotic or eukaryotic mRNA, genomic DNA sequences from procaryotic or eukaryotic DNA, and even synthetic DNA sequences. A transcription termination sequence will usually be located 3′ to the coding sequence.

Likewise, “encodes”, unless evident from its context, will be meant to include DNA sequences that encode a polypeptide, as the term is typically used, as well as DNA sequences that are transcribed into inhibitory antisense molecules.

The term “loss-of-function”, as it refers to genes inhibited by the subject RNAi method, refers a diminishment in the level of expression of a gene when compared to the level in the absence of dsRNA constructs.

The term “expression” with respect to a gene sequence refers to transcription of the gene and, as appropriate, translation of the resulting mRNA transcript to a protein. Thus, as will be clear from the context, expression of a protein coding sequence results from transcription and translation of the coding sequence.

“Cells,” “host cells” or “recombinant host cells” are terms used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

By “recombinant virus” is meant a virus that has been genetically aftered, e.g., by the addition or insertion of a heterologous nudeic acid construct into the particle.

As used herein, the terms “transduction” and “transfection” are art recognized and mean the introduction of a nucleic acid, e.g., an expression vector, into a recipient cell by nucleic acid-mediated gene transfer. “Transformation”, as used herein, refers to a process in which a cell's genotype is changed as a result of the cellular uptake of exogenous DNA or RNA, and, for example, the transformed cell expresses a dsRNA construct.

“Transient transfection” refers to cases where exogenous DNA does not integrate into the genome of a transfected cell, e.g., where episomal DNA is transcribed into mRNA and translated into protein.

A cell has been “stably transfected” with a nucleic acid construct when the nucleic acid construct is capable of being inherited by daughter cells.

As used herein, a “reporter gene construct” is a nucleic acid that includes a “reporter gene” operatively linked to at least one transcriptional regulatory sequence. Transcription of the reporter gene is controlled by these sequences to which they are linked. The activity of at least one or more of these control sequences can be directly or indirectly regulated by the target receptor protein. Exemplary transcriptional control sequences are promoter sequences. A reporter gene is meant to include a promoter-reporter gene construct that is heterologously expressed in a cell.

### DESCRIPTION OF THE SPECIFIC EMBODIMENTS

Methods and compositions for producing hairpin RNA expression modules, e.g., shRNA expression modules, for specific target nucleic acids are provided. In the subject methods, an initial nucleic acid, e.g., dsDNA, synthetic DNA, etc., corresponding to the target nucleic acid of interest is converted to an intermediate nucleic acid. The resultant intermediate nucleic acid is then converted to a linear dsDNA that includes at least one copy of the hairpin RNA expression module of interest, or a precursor (i.e., pro-shRNA expression module) thereof. Also provided are reagents, systems and kits for use in practicing the subject methods. The subject methods and compositions find use in a variety of different applications, including the production of shRNA molecules specific for target genes, and the production of libraries of shRNA molecules.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Methods recited herein may be carried out in any order of the recited events which is logically possible, as well as the recited order of events.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

In further describing the subject invention, the subject methods of producing shRNA encoding nucleic acids are described first in greater detail, followed by a description of the product nucleic acids produced thereby and a review of various representative applications, including research and therapeutic applications, in which the subject invention finds use. Finally, systems and kits that find use in practicing various aspects of the subject invention are discussed.

### Methods

As summarized above, the subject invention provides methods of efficiently producing hairpin RNA expression modules, e.g., shRNA expression modules, as well as libraries thereof, that encode hairpin RNAs, e.g., shRNAs, that are specific for a target nucleic acid(s). A feature of the subject methods is that an initial nucleic acid that corresponds to the target nucleic acid of the hairpin RNA to be produced is employed as a starting material. By corresponds is meant that the initial nucleic acid employed as “input” in the subject methods is one that includes a sequence found in the target nucleic acid. In many embodiments, the initial nucleic acid is a fragment of the target nucleic acid, as described in greater detail below.

Because the initial nucleic acid (which may be dsDNA in certain embodiments, as described in greater detail below) corresponds to the target nucleic acid, the product hairpin RNA (hRNA) expression modules that are produced from the initial dsDNA according to the subject methods encode hRNAs, e.g., shRNAs, that are specific for the target nucleic acid, because the expression modules include two encoding domains having sequences found in the target nucleic acid as provided by the initial nucleic acid. As such, a hRNA, e.g., shRNA, transcribed from the product hRNA encoding molecules or expression modules includes a double-stranded RNA domain having a sequence that is the RNA equivalent of a sequence found in the target nucleic acid.

In practicing the subject methods, the first step is to provide the initial nucleic acid for which the expression modules are to be prepared. In certain embodiments, the initial nucleic acid is a dsDNA molecule that includes a coding sequence for an mRNA or least a portion thereof. The dsDNA molecule that serves as the initial nucleic acid may be obtained using any convenient protocol. As such, the dsDNA molecule may be harvested from a naturally occurring source, e.g., it may be genomic DNA found in the nuclear fraction of a cell lysate, where any convenient means for obtaining such a fraction may be employed and numerous protocols for doing so are well known in the art. The genomic source may be genomic DNA representing the entire genome from a particular organism, tissue or cell type, as desired

In yet other embodiments, the target nucleic acid to which the initial dsDNA corresponds is a double-stranded cDNA molecule, e.g., that has been prepared from an mRNA of interest for which the to be produced hRNA, e.g., shRNA, is directed. cDNA may be prepared from an initial RNA source using any convenient protocol. Where desired, an initial RNA sample, e.g., mRNA sample, is subjected to a series of enzymatic reactions under conditions sufficient to ultimately produce double-stranded DNA for each initial mRNA in the initial sample. The initial RNA sample, e.g., total RNA sample or mRNA sample, will typically be derived from a physiological source. The physiological source may be derived from a variety of eukaryotic sources, with physiological sources of interest including sources derived from single-celled organisms such as yeast and multicellular organisms, including plants and animals, particularly mammals, where the physiological sources from multicellular organisms may be derived from particular organs or tissues of the multicellular organism, or from isolated cells derived therefrom. In obtaining the RNA preparation from the physiological source from which it is derived, any convenient protocol for isolation of total RNA from the initial physiological source may be employed. Methods of isolating RNA from cells, tissues, organs or whole organisms are known to those of skill in the art and include those described in Maniatis et al. (1989), Molecular Cloning: A Laboratory Manual 2d Ed. (Cold Spring Harbor Press).

In converting an initial RNA sample to cDNA, the first step is typically to contact with RNA sample with a primer for first strand cDNA synthesis, e.g., a first strand cDNA primer. As is known in the art, the primer may be a poly dT primer, a random primer or gene specific primer, depending on the nature of the product cDNA sample that is desired. Contact of the RNA sample with the primer(s) results in the production of primer-mRNA hybrid molecules. Conversion of primer-mRNA hybrids to double-stranded cDNA by reverse transcriptase proceeds through an RNA:DNA intermediate which is formed by extension of the hybridized promoter-primer by the RNA-dependent DNA polymerase activity of reverse transcriptase. The RNaseH activity of the reverse transcriptase then hydrolyzes at least a portion of the RNA:DNA hybrid, leaving behind RNA fragments that can serve as primers for second strand synthesis (Meyers et al., Proc. Nat'l Acad. Sci. USA (1980) 77:1316 and Olsen & Watson, Biochem. Biophys. Res. Commun. (1980) 97:1376). Extension of these primers by the DNA-dependent DNA polymerase activity of reverse transcriptase results in the synthesis of double-stranded cDNA. Other mechanisms for priming of second strand synthesis may also occur, including “self-priming” by a hairpin loop formed at the 3′ terminus of the first strand cDNA (Efstratiadis et al. (1976), Cell 7, 279; Higuchi et al. (1976), Proc. Natl, Acad, Sci USA 73, 3146; Maniatis et al. (1976), Cell 8, 163; and Rougeon and Mach (1976), Proc. Natl. Acad. Sci. USA 73, 3418; and “non-specific priming” by other DNA molecules in the reaction, i.e. the promoter-primer.

Alternatively, the initial nucleic acid may be a synthetic nucleic acid. For example, where the sequence of the target nucleic acid is known at least partially, the dsDNA molecule may be produced synthetically, e.g., by using known in the art nucleic acid synthesis protocols (such as protocols based on phosphoramidite chemistry, etc.).

As such, the initial nucleic acid that serves as “input” in the subject methods may be a single nucleic acid or plurality of distinct nucleic acids, including a complex mixture of nucleic acids, where the nucleic acid(s) may be genomic DNA, cDNA, etc.

While in certain embodiments the target nucleic acid, if present as a dsDNA molecule, may be used directly as the initial nucleic acid in the subject methods, where desired, the target nucleic acids are size modified to produce a suitable initial dsDNA for use in the subject methods. As such, in representative embodiments, the first step of the subject methods is to fragment the target nucleic acid into a plurality of fragments. In other words, in certain embodiments it may be desirable to fragment the target dsDNA molecule, e.g., cDNA, into a plurality of different fragments or pieces, which fragments or pieces are suitable to serve as the initial dsDNA molecules for the subject methods. By plurality is meant at least 2, usually at least about 5, and more usually at least about 10, where the number of distinct fragments produced from a given parent dsDNA molecule in the subject methods will often depend on the length of the parent dsDNA molecule, but may be as high as about 25 or higher, e.g., about 35 or higher. The resultant fragment product molecules in many embodiments range in length from about 20 to about 100 bp, e.g., from about 25 to about 80 bp. In yet other embodiments, no fragmentation is performed, e.g., where longer hRNA expression modules are the desired product.

When desired, fragmentation of a target nucleic acid may be accomplished using any convenient protocol, where protocols of interest include both mechanical/physical protocols and chemical, e.g., enzymatic, protocols. For example, the initial dsDNA molecules may be subjected to physical conditions that shear or mechanically break up the initial dsDNA molecules in to fragments of appropriate size. DNA shearing protocols are well known to those of skill in the art. Alternatively, the dsDNA molecules may be fragmented into desired size ranges by employing a chemical reagent, e.g., an enzymatic reagent, that cleaves the dsDNA molecule into fragments of desired size.

In many embodiments, an enzymatic cleavage protocol is employed, in which the target molecule is contacted with one or more nucleases, e.g., restriction endonucleases, which cleave the dsDNA molecule into fragments of desired size.

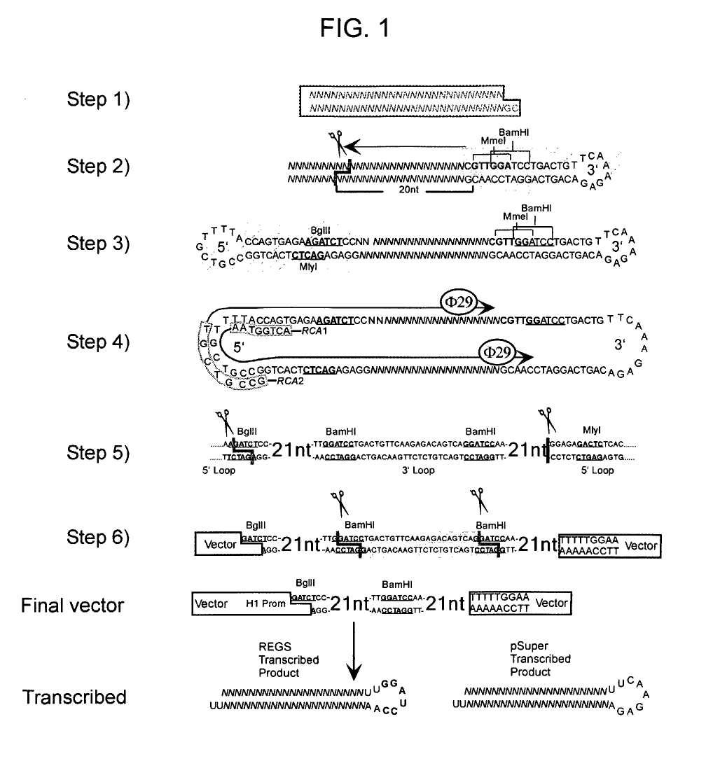

In certain embodiments, a single frequently cutting enzyme may be employed, such as CVIJI or DNAse. In certain embodiments, a combination of two or more restriction endonulceases are employed, where the two or more restriction endonucleases that are employed are selected or chosen to cleave the dsDNA molecule into fragments of a predetermined size. In such embodiments, the number of restriction endonucleases that are employed may vary, e.g., from about 2 to about 10, such as from about 3 to about 8, including from about 3 to about 7, e.g., 3, 4, 5 or 6. In these embodiments, the plurality of restriction endonucleases are chosen based on the predicted frequency of their respective recognition sites in the dsDNA to be cleaved, so that the combined action of the plurality of nucleases at least theoretically results in fragments of a desired predetermined size. As such, a collection or plurality of endonucleases may be chosen that at least theoretically will cleave the target nucleic acid into fragments that have a predicted predetermined size ranging from about 10 to about 50 bp, such as from about 15 to about 35 bp, including from about 19 to about 29 bp, e.g., 19 bp, 20 bp, 21 bp, 22 bp or 23 bp. As desired, the collection or plurality of restriction endonucleases may also be chosen to provide for fragments that include the same single-stranded overhang, where the overhang (when present) may range from about 1 to about 6 nt or longer, such as from about 1 to about 5 nt, including from about 2 to about 4 nt. The overhang may have any convenient sequence, e.g., GC, etc. In these embodiments, depending on the desired parameters for the fragments to be produced, e.g., size, presence of overhang etc., the collection or plurality of endonucleases that is employed may vary greatly, where suitable collections or combinations of enzymes can readily be determined by those of skill in the art based on known recognition sites, predicted frequency in the dsDNA to be cleaved, etc. A representative enzyme collection that finds use includes the specific representative enzyme collection made up of Hinpl, BsaHl, Acil, Hpall, HpyCHIV, and Taq∝l employed in the experimental section, below, as well as in step 1 of FIG. 1.

In the above embodiments where the initial nucleic acid is a dsDNA, following provision of the initial dsDNA molecule and any desired fragmentation thereof, the next step in the subject methods is to convert the initial dsDNA to a single-stranded nucleic acid intermediate that includes a linker domain, e.g., 3′ loop domain, flanked by intra-complementary domains that are the strands of the initial dsDNA molecule, where the intermediate nucleic acid can assume a hairpin configuration and therefore may be referred to a hairpin intermediate nucleic acid. The resultant intermediate nucleic acid is a single stranded molecule that may assume a configuration that includes a single stranded loop structure and a double-stranded stem structure, such that the nucleic acid has an overall hairpin configuration. The length of the single stranded loop structure may vary, but in certain embodiments ranges from about 6 to about 20 nt, such as from about 7 to about 15 nt, including from about 8 to about 10 nt. The length of the stem component may be the same as or longer than the length of the initial dsDNA from which the intermediate is produced, but in many embodiments ranges from about 2 to about 50 bp, including from about 5 to about 25 bp.

The hairpin intermediate may be produced by combining the initial dsDNA with a linker nucleic acid, such as a pro-3′ loop nucleic acid, under ligation conditions, such that the linker nucleic acid, e.g., the pro-3′ loop nucleic acid, ligates to the dsDNA to produce the desired intermediate. In many embodiments, the linker nucleic acid is a single stranded nucleic acid, e.g., DNA, that includes 5′ and 3′ complementary domains separated by a loop domain. In these embodiments, the 5′ and 3′ complementary domains hybridize to each other to produce a hairpin structure having a double-stranded stem domain and single stranded loop domain. Where the linker nucleic acid is to be ligated to a dsDNA having an overhang, e.g., GC, the double-stranded stem domain will end in a complementary overhang, e.g., CG.

Depending on the particular protocol being practiced, the protocol may include intermediate size modification step, as described in greater detail below. In such embodiments, the double-stranded stem domain of the pro linker nucleic acid may include a suitable size modification restriction endonuclease recognition site, where such a site will typically be positioned near the end of the linker nucleic acid that is to be ligated to the dsDNA (i.e., where both the 5′ and 3′ ends are positioned), e.g., within about 5 bp, within about 3 bp, within about 2 bp of the stem terminus. In these embodiments, the restriction endonuclease recognition site is conveniently a site that is recognized by an endonuclease that cleaves a dsDNA at a defined distance from the site, where the defined distance may range from about 10 to about 40 bp, such as from about 15 to about 30 bp, e.g., 18 bp, 19 bp, 20 bp, 21 bp, 22 bp, 23 bp, etc. Representative sites of interest include, but are not limited to, sites recognized by the following restriction endonucleases: Mmel, and the like. In yet other embodiments where longer hRNA expression modules are the desired product, this size modification step is not performed.

In certain embodiments, e.g., where it is desired to size modify the loop domain of an pro-expression module of a product shRNA encoding nucleic acid, as described in greater detail below, the double-stranded stem domain of the linker nucleic acid may further include at least one additional restriction endonuclease recognition site, where representative sites of interest include, but are not limited to, sites recognized by the following endonucleases: BamHl, and the like.

In this step of the subject methods, the linker nucleic acid may be ligated to the initial dsDNA using any convenient protocol. Typically, the linker nucleic acid is combined with the dsDNA in the presence of a suitable ligase, e.g., T4 DNA ligase, E. coli DNA ligase, etc., and maintained under suitable ligation conditions, where such conditions are well-known.

In yet other embodiments, the intermediate nucleic acid is prepared from a purely synthetic initial single-stranded nucleic acid, or collection of initial single-stranded nucleic acids. In certain of these embodiments, a library of molecules having a random 5′ domain linked to a common linker domain is employed as the initial or input nucleic acid. The random 5′ domain has a length that is of interest for an siRNA coding region, such as from about 15 to about 35 bp, including from about 19 to about 29 bp, e.g., 19 bp, 20 bp, 21 bp, 22 bp or 23 bp. In this embodiment, the random 5′ domain of the molecules that make up the library is linked or bonded to a 3′ linker domain, where this domain is analogous to the linker domain described above. As such, the libraries in these embodiments are made up of a large number of distinct nucleic acids of different sequence with respect to their random 5′ domain and common sequence with respect to their 3′ domain, where the number of distinct nucleic acids of differing random domain in the library may range from about 4¹⁵ to about 435, including from about 4¹⁹ to about 4²⁹, e.g., 4¹⁹, 4²⁰, 4²¹, 4²², or 4²³ Initial nucleic acids of these embodiments may readily be converted to intermediate nucleic acids using primer extension protocols, with the common 5′ linker domain (having a hairpin configuration) serving as a double-stranded primer site and the single stranded random domain serving as the template strand.

Following production of the intermediate nucleic acid (e.g., from the dsDNA fragment of the target nucleic acid of interest or a library of synthetically produced initial nucleic acids, as reviewed above), the resultant intermediate may be size modified, as desired. For example, where the initial dsDNA molecule to which the linker nucleic acid is ligated is longer than the desired length for product shRNA molecule, e.g., longer than about 30 bp, such as longer than about 25 bp, the intermediate hairpin nucleic acid may be size modified to shorten its length to one that ultimately provides shRNA molecules of the appropriate size, e.g., from about 17 to about 23 nt, including from about 19 to about 21 or 22 nt, as described in greater detail below. In certain embodiments, a size modification enzyme, such as Mmel as described above, is employed in this optional step of the subject methods. As indicated above, in other embodiments this size modification step is not performed. For example, where expression modules that encode longer hRNA molecules, e.g., longer than about 35 bp, such as 40 bp or longer, 50 bp or longer, 75 bp or longer, 100 bp or longer, etc., the size modification step is not performed.

The next step of the subject methods is to convert the intermediate, e.g., hairpin intermediate, nucleic acid into a linear ds DNA molecule that includes at least one hRNA, e.g., shRNA, expression module or precursor thereof, i.e., pro-hRNA, e.g., shRNA, expression module, where the shRNA expression module is made up of a hairpin encoding domain flanked by siRNA encoding domains. In this conversion step, the intermediate nucleic acid, which has a single-stranded hairpin configuration, such as is shown in step 2 of FIG. 1, is converted to a linear double-stranded DNA molecule. This conversion step may include a variety of different specific protocols, where the protocols may or may not include an amplification step, as may be desired.

In one representative conversion protocol, an amplification step is not included. In this representative protocol, the intermediate nucleic acid is contacted with a suitable primer, e.g., that hybridizes to a universal priming site ligated onto the terminus of the molecule, a polymerase and the appropriate deoxynucleotides (i.e., dGTP, dCTP, dATP and dTTP) and maintained under primer extension conditions such that the a second strand DNA is synthesized under a template dependent primer extension reaction, where the intermediate molecule has been disassociated and serves as the template strand. In this particular protocol, one double-stranded product is produced for each initial intermediate molecule. As such, this protocol is representative of a non-amplification conversion protocols. Primer extension reaction conditions and reagents employed therein, e.g., polymerases, buffers, etc., are well known in the art and need not be described in greater detail here.

In other embodiments, it is desirable to employ a conversion protocol that includes amplification, such that amplified amounts of product linear ds DNA molecules are produced for an initial intermediate molecule. Any convenient amplification conversion protocol may be employed. One representative amplification conversion protocol is a polymerase chain reaction (PCR) protocol, in which forward and reverse priming sites are ligated onto the end of the intermediate molecule, where the product of this ligation is then contacted with appropriate forward and reverse primers, a suitable polymerase and the appropriate deoxynucleotides to produce a PCR reaction mixture, which PRC reaction mixture is then subjected to polymerase chain reaction (PCR conditions). The polymerase chain reaction (PCR) is well known in the art, being described in U.S. Pat. Nos.: 4,683,202; 4,683,195; 4,800,159; 4,965,188 and 5,512,462, the disclosures of which are herein incorporated by reference. By polymerase chain reaction conditions is meant the total set of conditions used in a given polymerase chain reaction, e.g. the nature of the polymerase or polymerases, the type of buffer, the presence of ionic species, the presence and relative amounts of dNTPs, etc. Using a suitable PCR protocol, multiple copies of a desired linear dsDNA molecule that includes an shRNA expression module or precursor thereof may be produced from a single intermediate molecule.

Yet another representative amplification conversion protocol of interest is a protocol that employs “rolling circle amplification.” In these rolling circle amplification protocols, the intermediate nucleic acid is first converted to a single stranded circular DNA molecule, i.e., a dumbbell configured template molecule. The circular single-stranded molecule serves as a template for geometric rolling circle amplification, in which forward and reverse rolling circle primers are contacted with the circular template under rolling circle amplification conditions sufficient to produce long complementary DNA strands that, upon hybridization to each other, include multiple copies of the desired shRNA expression module or precursor thereof. Rolling circle amplification conditions are known in the art and described in, among other locations, U.S. Pat. Nos. 6,576,448; 6,287,824; 6,235,502; and 6,221,603; the disclosures of which are herein incorporated by reference.

In these protocols, the single stranded circular template molecule may be produced from the intermediate nucleic acid by ligating the 5′ and 3′ ends of the intermediate nucleic acid to a second linker nucleic acid, e.g., a pro-5′ loop nucleic acid, which ligation reaction produces a suitable singled-stranded circular template, such as the dumbbell configured template depicted in step 3 of FIG. 1. In many embodiments, the pro-5′ loop nucleic acid that is ligated to the 3′ loop containing DNA is one that includes suitable rolling circle amplification primer sites, as well as restriction endonuclease recognition sites for use in excising desired shRNA expression modules from the product dsDNA produced by the rolling circle amplification process. For example, the pro-5′ loop nucleic acid may include recognition sites for two different endonucleases, such that in the rolling circle amplification product, each shRNA expression module is flanked by two different restriction endonuclease sites, which sites provide for convenient excision of each expression module from the rolling circle amplification product. For example, the pro-5′ loop employed in the representative protocol depicted in FIG. 1 includes a recognition site for Bglll and Mlyl positioned in the loop structure such that, following rolling circle amplification, each expression module is bounded on one side by the Bglll recognition site and on the other side by the Mlyl recognition site. Depending on the features present in the pro-5′ loop nucleic acid, the length of the pro-5′ loop strand may vary, but in many embodiments range from about 20 to about 150 nt, such as from about 40 to about 100 nt.

For rolling circle amplification, the circular template strand is contacted with forward and reverse primers, a suitable polymerase, and the four dNTPs, as well as any other desired reagents to produce a rolling circle amplification reaction mixture, which reaction mixture is then maintained under rolling circle amplification conditions. In certain embodiments, the polymerase that is employed is a highly processive polymerase. By highly processive polymerase is meant a polymerase that elongates a DNA chain without dissociation over extended lengths of nucleic acid, where extended lengths means at least about 50 nt long, such as at least about 100 nt long or longer, including at least about 250 nt long or longer, at least about 500 nt long or longer, at least about 1000 nt long or longer. In many embodiments, the polymerase employed in the amplification step is a phage polymerase. Of interest in certain embodiments is the use of a φ29-type DNA polymerase. By φ29-type DNA polymerase is meant either: (i) that phage polymerase in cells infected with a φ29-type phage; (ii) a φ29-type DNA polymerase chosen from the DNA polymerases of phages φ29, Cp-1, PRD1, φ15, φ21, PZE, PZA, Nf, M2Y, B103, SF5, GA-1, Cp-5, Cp-7, PR4, PR5, PR722, and L17; or (iii) a φ29-type polymerase modified to have less than ten percent of the exonuclease activity of the naturally-occurring polymerase, e.g., less than one percent, including substantially no, exonuclease activity. Representative φ29 type polymerases of interest include, but are not limited to, those polymerases described in U.S. Pat. No. 5,198,543, the disclosure of which is herein incorporated by reference.

The above described conversion step results in the production of linear dsDNA molecules that include at least one shRNA expression module or precursor thereof, where the resultant dsDNA molecules may or may not include more than one shRNA expression modules, depending on the particular conversion protocol that is employed. For example, in the representative non-amplification conversion protocol and PCR amplification conversion protocol described above, the product linear dsDNA molecules include a single shRNA expression module. In contrast, in the representative rolling circle amplification protocol described above, the product dsDNA molecule includes multiple copies of the desired shRNA expression module, where each copy is separated from each other by a domain corresponding to a linker domain, e.g., the 5′ loop nucleic acid employed to produce the circular template molecule.

A feature of the product linear dsDNA molecules produced by the conversion step of the subject methods is that they include at least one hRNA, e.g., shRNA, expression module or precursor thereof (i.e., pro-shRNA expression module). By hRNA expression module is meant a stretch or domain of double stranded DNA that can be transcribed into an hRNA molecule, and in particular a hairpin RNA molecule that acts as an interfering RNA agent, i.e., an RNAi agent. The hRNA expression module includes a linker domain flanked by siRNA encoding domains. The linker domain is a domain that is transcribed under appropriate conditions into the single-stranded loop, e.g., a 3′ single stranded loop, of a hRNA molecule. In certain embodiments, the length of this domain may range from about 5 to about 20 bp, such as from about 5 to about 15 bp. In pro-hRNA expression modules, the sequence of this domain may be longer, ranging from about 5 to about 100 bp, including from about 10 to about 50 bp.

The flanking siRNA encoding domains each have sequences that are transcribed into one strand of the self-complementary stem portion of a hRNA, e.g., shRNA, molecule. As such, the flanking siRNA encoding domains have the same sequence in opposing orientations. The length of the siRNA encoding domains may vary, an in representative embodiments ranges from about 17 to about 30 bp, including from about 19 to about 25 bp, e.g., such as a 19, 20 or 21 bp encoding domain. In yet other embodiments, the length of these domains is longer than about 30 bp, such as longer than about 45 bp, e.g., longer than about 50 bp, such as 75 bp or longer, 100 bp or longer, 200 bp or longer, etc.

Where desired, and depending on the particular application in which the subject methods are employed, the expression module may be excised from the product linear dsDNA molecule and cloned into a suitable vector. Representative vectors into which the expression module may be cloned include, but are not limited to: plasmids; viral vectors; and the like.

Representative eukaryotic plasmid vectors of interest include, for example: pCMVneo, pShuttle, PDNR and Ad-X (Clontech Laboratories, Inc.); as well as BPV, EBV, vaccinia, SV40, 2-micron circle, pcDNA3.1, pcDNA3.1/GS, pYES2/GS, pMT, p IND, pIND(Spl), pVgRXR, and the like, or their derivatives. Such plasmids are well known in the art (Botstein et al., Miami Wntr. SyTnp. 19:265-274, 1982; Broach, In: “The Molecular Biology of the Yeast Saccharomyces: Life Cycle and Inheritance”, Cold Spring Harbor Laboratory, Cold Spring Harbor, NY, p. 445-470, 1981; Broach, Cell 28:203-204, 1982; Dilon et at., J. Clin. Hematol. Oncol. 10:39-48, 1980; Maniatis, In: Cell Biology: A Comprehensive Treatise, Vol. 3, Gene Sequence Expression, Academic Press, NY, pp. 563-608, 1980.

A variety of viral vector delivery vehicles are known to those of skill in the art and include, but are not limited to: adenovirus, herpesvirus, lentivirus, vaccinia virus and adeno-associated virus (AAV).

In those embodiments where the expression module is to be transcribed into an shRNA molecule from the vector on which the expression module resides, the expression module will be operably linked to a suitable promoter on the vector. In general, any convenient promoter may be employed, so long as the promoter can be activated in the desired environment to transcribe expression module and produce the desired shRNA molecule. Promoters of interest include both constitutive and inducible promoters. Exemplary promoters for use in the present invention are selected such that they are functional in the cell type (and/or animal or plant) into which they are being introduced. Representative specific promoters of interest include, but are not limited to: pol lll promoters (such as mammalian (e.g., mouse or human) U6 and H1 promoters, VA1 promoters, tRNA promoters, etc.); pol II promoters; inducible promoters, e.g., TET inducible promoters; bacteriophage RNA polymerase promoters, e.g., T7, T3 and Sp6, and the like. Other promoters known in the art may also be employed, where the particular promoters chosen will depend, at least in part, on the environment in which expression is desired.

In certain embodiments, a plurality, e.g., 2 or more, 3 or more, 4 or more, 5 or more, such as 7, 8, 9, 10 or more, distinct expression modules may be cloned into the same vector. For example, the 5′ loop described above could selected to encode a small promoter. In such embodiments, after the rolling circle amplification, the resultant products could be digested to release the individual cassettes then religated into a concatemer structure. This approach could be performed so as to achieve a “shuffling” of the cassettes. The resultant concatemer of a plurality of cassettes could then be cloned into a vector to provide a vector expressing multiple shRNAs.

Where desired, the methods may include a step of size modifying the linking domain of a pro- hRNA expression module. One convenient protocol includes employing built in restriction sites to excise a region or portion of the linking domain, as shown in step 6 of FIG. 1, where the “built-in” restriction sites are present by proper selection of a linker nucleic acid. This size modification step may be employed either before or after the pro-expression module is cloned into a vector, as desired. When employed, the size of the linking domain of the pro-expression module may be reduced by from about 5 to about 90 bp, including from about 10 to about 50 bp.

The above methods result in the production of a hRNA expression module, e.g., a shRNA expression module, i.e., a shRNA encoding double stranded nucleic acid, which may or may not be present on a vector. A feature of the subject method is that it can readily produce multiple distinct hRNA, e.g., shRNA, expression modules that each encode a different hRNA molecule for the same target nucleic acid sequence. Thus, in certain embodiments the subject methods result in the production of multiple different hRNA encoding nucleic acids for the same target nucleic acid.

In certain embodiments, the subject methods are employed to rapidly produce at least one, and typically multiple, hRNA encoding nucleic acids for a plurality of different target nucleic acids. For example, the subject methods may be employed to produce a library of shRNA encoding nucleic acids by employing multiple distinct target nucleic acids as “input” for the methods, where the multiple distinct “input” target nucleic acids may be in the form of a cDNA library, genomic library etc. As such, in certain embodiments the subject methods result in the production of an shRNA encoding nucleic acid library, where the library may be a library for given organism, tissue type, cell type, or fraction thereof, depending on the nature of the “input” target nucleic acid composition.

A feature of the libraries produced by the subject methods is that they can be highly complex, by which is meant that they can include large number of individual shRNA encoding nucleic acids (i.e., expression modules) that each encode a different shRNA molecule of distinct or different sequence. As such, the complexity of the subject libraries (in terms of numbers of distinct shRNA expression modules) can be 1×10² or more, 1×10³ or more, 1×10⁴ or more, 1×10⁵ or more, 1×10⁶ or more, where the complexity of the product library is primarily a factor of the complexity of the input nucleic acid. A feature of the subject libraries is that the complexity and bias of the libraries is determined by the input nucleic acid. As indicated above, the input nucleic acid may be genomic DNA, a cDNA library (which may or may not be normalized), etc., such that in certain embodiments the product library may span an entire genome. Because of the nature of the subject methods, the library may include shRNA expression modules that produce shRNAs directed to both known and unknown genes, since knowledge of a gene is not required by the subject methods to produce a shRNA to that gene. Another feature of certain embodiments of the subject libraries is that they include a high percentage of expression modules that encode an shRNA molecule of appropriate size, as described above, where the number percent of such modules may be as high as 85% or higher, e.g., 90%, 95%, etc. or higher. In certain embodiments, the libraries include aproximately equal numbers of expression modules that encode the desired shRNA molecules in the sense orientation, while the remainder of the modules encode their shRNA molecules in the antisense orientiation, where the ratio of sense to antisense orientations in the product libraries may range from about 30/70 to about 70/30, such as from about 40/60 to about 60/40, including from about 45/55 to about 55/45, e.g., about 50/50. An important feature of the subject methods is that they can rapidly produce highly complex libraries of shRNA encoding nucleic acids, as described above. By rapidly produce is meant that the subject libraries can be produced by a single practioner a less than about 15 days, such as less than about 10 days, including less than about 5 days, e.g., 4 days or less.

### Utility

The product hRNA, e.g., shRNA, encoding dsDNA molecules produced by the above described methods find use in a variety of applications, particularly where the production of shRNA molecules is desired. For example, applications in which the production of shRNA molecules is desired include applications in which it is desired to modulate expression of a target gene or genes in a cell or host including such a cell harboring such a target gene. In many such applications, the shRNA encoding constructs and shRNA products thereof are employed to reduce target gene expression of one or more target genes in a cell or organism. By reducing expression is meant that the level of expression of a target gene or coding sequence is reduced or inhibited by at least about 2-fold, usually by at least about 5-fold, e.g., 10-fold, 15-fold, 20-fold, 50-fold, 100-fold or more, as compared to a control. By modulating expression of a target gene is meant altering, e.g., reducing, transcription/translation of a coding sequence, e.g., genomic DNA, mRNA etc., into a polypeptide, e.g., protein, product. As such, the subject invention provides methods of reducing or inhibiting expression of one or more target genes in a cell or organism.

In general, applications in which the shRNA constructs and shRNA products thereof find use include transcribing an shRNA molecule from the shRNA expression module present on the dsDNA product of the subject methods. For transcription, the expression module under the control of a suitable promoter is maintained in an environment in which the promoter directs transcription of its operatively linked expression module.

Production of the shRNA encoded molecules may occur in a cell free environment or inside of a cell. Where production of the shRNA product molecules is desired to occur inside of a cell, any convenient method of delivering the construct to the target cell may be employed. Where it is desired to express the shRNA encoded molecules inside of a cell, the above expression module, e.g., under the control of a suitable promoter, is introduced into the target cell. Any convenient protocol may be employed, where the protocol may provide for in vitro or in vivo introduction of the construct into the target cell, depending on the location of the target cell.

For example, where the target cell is an isolated cell, the construct may be introduced directly into the cell under cell culture conditions permissive of viability of the target cell, e.g., by using standard transformation techniques. Such techniques include, but are not necessarily limited to: viral infection, transformation, conjugation, protoplast fusion, electroporation, particle gun technology, calcium phosphate precipitation, direct microinjection, viral vector delivery, and the like. The choice of method is generally dependent on the type of cell being transformed and the circumstances under which the transformation is taking place (i.e. in vitro, ex vivo, or in vivo). A general discussion of these methods can be found in Ausubel, et al, Short Protocols in Molecular Biology, 3rd ed., Wiley & Sons, 1995.

Alternatively, where the target cell or cells are part of a multicellular organism, the construct may be administered to the organism or host in a manner such that the construct is able to enter the target cell(s), e.g., via an in vivo or ex vivo protocol. By “in vivo,” it is meant that the target construct is administered to a living body of an animal. By “ex vivo” it is meant that cells or organs are modified outside of the body. Such cells or organs are typically returned to a living body. Methods for the administration of nucleic acid constructs are well known in the art. Nucleic acid constructs can be delivered with cationic lipids (Goddard, et al, Gene Therapy, 4:1231-1236, 1997; Gorman, et al, Gene Therapy 4:983-992, 1997; Chadwick, et al, Gene Therapy 4:937-942, 1997; Gokhale, et al, Gene Therapy 4:1289-1299, 1997; Gao, and Huang, Gene Therapy 2:710-722, 1995,), using viral vectors (Monahan, et al, Gene Therapy 4:40-49, 1997; Onodera, et al, Blood 91:30-36, 1998,), by uptake of “naked DNA”, and the like. Techniques well known in the art for the transformation of cells (see discussion above) can be used for the ex vivo administration of nucleic acid constructs. The exact formulation, route of administration and dosage can be chosen empirically. (See e.g. Fingl et al., 1975, in “The Pharmacological Basis of Therapeutics”, Ch. 1 pI).

As such, in certain embodiments the expression module, which may be present on a vector, (e.g., plasmids, viral vectors, etc) is administered to a multicellular organism that includes the target cell. By multicellular organism is meant an organism that is not a single celled organism. Multicellular organisms of interest include animals, where animals of interest include vertebrates, where the vertebrate is a mammal in many embodiments. Mammals of interest include; rodents, e.g. mice, rats; livestock, e.g. pigs, horses, cows, etc., pets, e.g. dogs, cats; and primates, e.g. humans.

The selected route of administration of the expression module to the multicellular organism depends on several parameters, including: the nature of the vectors that carry the expression module, the nature of the delivery vehicle, the nature of the multicellular organism, and the like. In certain embodiments, linear or circularized DNA, e.g. a plasmid, is employed as the vector for delivery of the expression module to the target cell. In such embodiments, the plasmid may be administered in an aqueous delivery vehicle, e.g., a saline solution. Alternatively, an agent that modulates the distribution of the vector in the multicellular organism may be employed. For example, where the vectors comprising the subject system components are plasmid vectors, lipid based, e.g. liposome, vehicles may be employed, where the lipid based vehicle may be targeted to a specific cell type for cell or tissue specific delivery of the vector. Patents disclosing such methods include: U.S. Pat. Nos. 5,877,302; 5,840,710; 5,830,430; and 5,827,703, the disclosures of which are herein incorporated by reference. Alternatively, polylysine based peptides may be employed as carriers, which may or may not be modified with targeting moieties, and the like. (Brooks, A. I., et al. 1998, J. Neurosci. Methods V. 80 p: 137-47; Muramatsu, T., Nakamura, A., and H. M. Park 1998, Int. J. Mol. Med. V. 1 p: 55-62). In yet other embodiments, the construct may be incorporated onto viral vectors, such as adenovirus derived vectors, sindbis virus derived vectors, retroviral derived vectors, etc. hybrid vectors, and the like, as described above. The above vectors and delivery vehicles are merely representative. Any vector/delivery vehicle combination may be employed, so long as it provides for the desired introduction of the expression module in into the target cell.

As such, in vivo and in vitro gene therapy delivery of the expression constructs according to the present invention is also encompassed by the present invention. In vivo gene therapy may be accomplished by introducing the expression module into cells via local injection of a polynucleotide molecule or other appropriate delivery vectors. (Hefti, J. Neurobiology, 25:1418-1435, 1994). For example, a polynucleotide molecule including the construct may be contained in an adeno-associated virus vector for delivery to the targeted cells (See for e.g., International Publication No. WO 95/34670; International Application No. PCT/US95/07178). The recombinant adeno-associated virus (AAV) genome typically contains AAV inverted terminal repeats flanking a DNA sequence that includes the construct.

Alternative viral vectors include, but are not limited to, retrovirus, adenovirus, herpes simplex virus and papilloma virus vectors. U.S. Pat. No. 5,672,344 (issued Sep. 30, 1997, Kelley et al., University of Michigan) describes an in vivo viral-mediated gene transfer system involving a recombinant neurotrophic HSV-1 vector. U.S. Pat. No. 5,399,346 (issued Mar. 21, 1995, Anderson et al., Department of Health and human Services) provides examples of a process for providing a patient with a therapeutic protein by the delivery of human cells which have been treated in vitro to insert a DNA segment encoding a therapeutic protein. Additional methods and materials for the practice of gene therapy techniques are described in U.S. Pat. No. 5,631,236 (issued May 20, 1997, Woo et al., Baylor College of Medicine) involving adenoviral vectors; U.S. Pat. No. 5,672,510 (issued Sep. 30, 1997, Eglitis et al., Genetic Therapy, Inc.) involving retroviral vectors; and U.S. Pat. No. 5,635,399 (issued Jun. 3, 1997, Kriegler et al., Chiron Corporation) involving retroviral vectors expressing cytokines.

Nonviral delivery methods include liposome-mediated transfer, naked DNA delivery (direct injection), receptor-mediated transfer (ligand-DNA complex), electroporation, calcium phosphate precipitation and microparticle bombardment (e.g., gene gun). Gene therapy materials and methods may also include inducible promoters, tissue-specific enhancer-promoters, DNA sequences designed for site-specific integration, DNA sequences capable of providing a selective advantage over the parent cell, labels to identify transformed cells, negative selection systems and expression control systems (safety measures), cell-specific binding agents (for cell targeting), cell-specific internalization factors, transcription factors to enhance expression by a vector as well as methods of vector manufacture. Such additional methods and materials for the practice of gene therapy techniques are described in U.S. Pat. No. 4,970,154 (issued Nov. 13, 1990, D. C. Chang, Baylor College of Medicine) electroporation techniques; International Application No. WO 9640958 (published 961219, Smith et al., Baylor College of Medicine) nuclear ligands; U.S. Pat. No. 5,679,559 (issued Oct. 21, 1997, Kim et al., University of Utah Research Foundation) concerning a lipoprotein-containing system for gene delivery; U.S. Pat. No. 676,954 (issued Oct. 14, 1997, K. L. Brigham, Vanderbilt University involving liposome carriers; U.S. Pat. No. 5,593,875 (issued Jan. 14, 1997, Wurm et al., Genentech, Inc.) concerning methods for calcium phosphate transfection; and U.S. Pat. No. 4,945,050 (issued Jul. 31, 1990, Sanford et al., Cornell Research Foundation) wherein biologically active particles are propelled at cells at a speed whereby the particles penetrate the surface of the cells and become incorporated into the interior of the cells. Expression control techniques include chemical induced regulation (e.g., International Application Nos. WO 9641865 and WO 9731899), the use of a progesterone antagonist in a modified steroid hormone receptor system (e.g., U.S. Pat. No. 5,364,791), ecdysone control systems (e.g., International Application No. WO 9637609), and positive tetracycline-controllable transactivators (e.g., U.S. Pat. Nos. 5,589,362; 5,650,298; and 5,654,168).

Because of the multitude of different types of vectors and delivery vehicles that may be employed, administration may be by a number of different routes, where representative routes of administration include: oral, topical, intraarterial, intravenous, intraperitoneal, intramuscular, etc. The particular mode of administration depends, at least in part, on the nature of the delivery vehicle employed for the vectors which harbor the construct. In certain embodiments, the vector or vectors harboring the expression module are administered intravascularly, e.g. intraarterially or intravenously, employing an aqueous based delivery vehicle, e.g. a saline solution.

The above-described product shRNA encoding molecules and shRNA products produced therefrom find use in a variety of different applications. Representative applications include, but are not limited to: drug screening/target validation, large scale functional library screening, silencing single genes, silencing families of genes, e.g., ser/thr kinases, phosphatases, membrane receptors, etc., and the like. The subject constructs and products thereof also find use in therapeutic applications, as described in greater detail separately below.

One representative utility of the present invention is as a method of identifying gene function in an organism, especially higher eukaryotes using the product siRNA to inhibit the activity of a target gene of previously unknown function. Instead of the time consuming and laborious isolation of mutants by traditional genetic screening, functional genomics using the subject product siRNA determines the function of uncharacterized genes by employing the siRNA to reduce the amount and/or alter the timing of target gene activity. The product siRNA can be used in determining potential targets for pharmaceutics, understanding normal and pathological events associated with development, determining signaling pathways responsible for postnatal development/aging, and the like. The increasing speed of acquiring nucleotide sequence information from genomic and expressed gene sources, including total sequences for mammalian genomes, can be coupled with use of the product siRNA to determine gene function in a cell or in a whole organism. The preference of different organisms to use particular codons, searching sequence databases for related gene products, correlating the linkage map of genetic traits with the physical map from which the nucleotide sequences are derived, and artificial intelligence methods may be used to define putative open reading frames from the nucleotide sequences acquired in such sequencing projects.

A simple representative assay inhibits gene expression according to the partial sequence available from an expressed sequence tag (EST). Functional alterations in growth, development, metabolism, disease resistance, or other biological processes would be indicative of the normal role of the ESTs gene product.

The present invention to be used in high throughput screening (HTS) applications. For example, individual clones from the library can be replicated and then isolated in separate reactions, or the library is maintained in individual reaction vessels (e.g., a 96 well microtiter plate) to minimize the number of steps required to practice the invention and to allow automation of the process. Solutions containing the shRNA encoding molecules or product shRNAs thereof that are capable of inhibiting the different expressed genes can be placed into individual wells positioned on a microtiter plate as an ordered array, and intact cells/organisms in each well can be assayed for any changes or modifications in behavior or development due to inhibition of target gene activity.

The shRNA encoding molecules or shRNA products thereof can be fed directly to, injected into, the cell/organism containing the target gene. The shRNA encoding molecules or shRNA products may be directly introduced into the cell (i.e., intracellularly); or introduced extracellularly into a cavity, interstitial space, into the circulation of an organism, introduced orally, or may be introduced by bathing an organism in a solution containing the shRNA encoding molecules or shRNA products. Methods for oral introduction include direct mixing of nucleic acids with food of the organism. Physical methods of introducing nucleic, acids include injection directly into the cell or extracellular injection into the organism of a nudeic add solution. The shRNA encoding molecules or shRNA products thereof may be introduced in an amount which allows delivery of at least one copy per cell. Higher doses (e.g., at least 5, 10, 100, 500 or 1000 copies per cell) of constructs or products thereof may yield more effective inhibition; lower doses may also be useful for specific applications. Inhibition is sequence-specific in that nucleotide sequences corresponding to the duplex region of the RNA are targeted for genetic inhibition.

The function of the target gene can be assayed from the effects it has on the cell/organism when gene activity is inhibited. This screening could be amenable to small subjects that can be processed in large number, for example, tissue culture cells derived from invertebrates or invertebrates, mammals, especially primates, and most preferably humans.

If a characteristic of an organism is determined to be genetically linked to a polymorphism through RFLP or QTL analysis, the present invention can be used to gain insight regarding whether that genetic polymorphism might be directly responsible for the characteristic. For example, a fragment defining the genetic polymorphism or sequences in the vicinity of such a genetic polymorphism can be screened for its impact, e.g., by producing a shRNA molecule corresponding to the fragment in the organism or cell, and evaluating whether an alteration in the characteristic is correlated with inhibition.

The present invention is useful in allowing the inhibition of essential genes. Such genes may be required for cell or organism viability at only particular stages of development or cellular compartments. The functional equivalent of conditional mutations may be produced by inhibiting activity of the target gene when or where it is not required for viability. The invention allows addition of shRNA at specific times of development and locations in the organism without introducing permanent mutations into the target genome.

In situations where alternative splicing produces a family of transcripts that are distinguished by usage of characteristic exons, the present invention can target inhibition through the appropriate exons to specifically inhibit or to distinguish among the functions of family members. For example, a hormone that contained an alternatively spliced transmembrane domain may be expressed in both membrane bound and secreted forms. Instead of isolating a nonsense mutation that terminates translation before the transmembrane domain, the functional consequences of having only secreted hormone can be determined according to the invention by targeting the exon containing the transmembrane domain and thereby inhibiting expression of membrane-bound hormone.

### Therapeutic Applications

The subject shRNA encoding molecules or shRNA products thereof also find use in a variety of therapeutic applications in which it is desired to selectively modulate, e.g., one or more target genes in a host, e.g., whole mammal, or portion thereof, e.g., tissue, organ, etc, as well as in cells present therein. In such methods, an effective amount of the subject shRNA encoding molecules or shRNA products thereof is administered to the host or target portion thereof. By effective amount is meant a dosage sufficient to selectively modulate expression of the target gene(s), as desired. As indicated above, in many embodiments of this type of application, the subject methods are employed to reduce/inhibit expression of one or more target genes in the host or portion thereof in order to achieve a desired therapeutic outcome.

Depending on the nature of the condition being treated, the target gene may be a gene derived from the cell, an endogenous gene, a pathologically mutated gene, e.g. a cancer causing gene, one or more genes whose expression causes or is related to heart disease, lung disease, Alzheimer's disease, Parkinson's disease, diabetes, arthritis, etc.; a transgene, or a gene of a pathogen which is present in the cell after infection thereof, e.g., a viral (e.g., HIV-Human Immunodeficiency Virus; HBV-Hepatitis B virus; HCV-Hepatitis C virus; Herpes-simplex 1 and 2; Varicella Zoster (Chicken pox and Shingles); Rhinovirus (common cold and flu); any other viral form) or bacterial pathogen. Depending on the particular target gene and the dose of construct or siRNA product delivered, the procedure may provide partial or complete loss of function for the target gene. Lower doses of injected material and longer times after administration of siRNA may result in inhibition in a smaller fraction of cells.

The subject methods find use in the treatment of a variety of different conditions in which the modulation of target gene expression in a mammalian host is desired. By treatment is meant that at least an amelioration of the symptoms associated with the condition afflicting the host is achieved, where amelioration is used in a broad sense to refer to at least a reduction in the magnitude of a parameter, e.g. symptom, associated with the condition being treated. As such, treatment also includes situations where the pathological condition, or at least symptoms associated therewith, are completely inhibited, e.g. prevented from happening, or stopped, e.g. terminated, such that the host no longer suffers from the condition, or at least the symptoms that characterize the condition.

A variety of hosts are treatable according to the subject methods. Generally such hosts are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In many embodiments, the hosts will be humans.

The present invention is not limited to modulation of expression of any specific type of target gene or nucleotide sequence. Representative classes of target genes of interest include but are not limited to: developmental genes (e.g., adhesion molecules, cyclin kinase inhibitors, cytokines/lymphokines and their receptors, growth/differentiation factors and their receptors, neurotransmitters and their receptors); oncogenes (e.g., ABLI, BCLI, BCL2, BCL6, CBFA2, CBL, CSFIR, ERBA, ERBB, EBRB2, ETSI, ETS1, ETV6, FOR, FOS, FYN, HCR, HRAS, JUN, KRAS, LCK, LYN, MDM2, MLL, MYB, MYC, MYCLI, MYCN, NRAS, PIM 1, PML, RET, SRC, TALI, TCL3, and YES); tumor suppressor genes (e.g., APC, BRCA 1, BRCA2, MADH4, MCC, NF 1, NF2, RB 1, TP53, and WTI); and enzymes (e.g., ACC synthases and oxidases, ACP desaturases and hydroxylases, ADP-glucose pyrophorylases, ATPases, alcohol dehydrogenases, amylases, amyloglucosidases, catalases, cellulases, chalcone synthases, chitinases, cyclooxygenases, decarboxylases, dextrinases, DNA and RNA polymerases, galactosidases, glucanases, glucose oxidases, granule-bound starch synthases, GTPases, helicases, hemicellulases, integrases, inulinases, invertases, isomerases, kinases, lactases, Upases, lipoxygenases, lyso/ymes, nopaline synthases, octopine synthases, pectinesterases, peroxidases, phosphatases, phospholipases, phosphorylases, phytases, plant growth regulator synthases, polygalacturonases, proteinases and peptidases, pullanases, recombinases, reverse transcriptases, RUBISCOs, topoisomerases, and xylanases); chemokines (e.g. CXCR4, CCR5), the RNA component of telomerase, vascular endothelial growth factor (VEGF), VEGF receptor, tumor necrosis factors nuclear factor kappa B, transcription factors, cell adhesion molecules, Insulin-like growth factor, transforming growth factor beta family members, cell surface receptors, RNA binding proteins (e.g. small nudeolar RNAs, RNA transport factors), translation factors, telomerase reverse transcriptase); etc.

As indicated above, the shRNA encoding molecules or shRNA thereof can be introduced into the target cell(s) using any convenient protocol, where the protocol will vary depending on whether the target cells are in vitro or in vivo.

Where the target cells are in vivo, the shRNA encoding molecules or shRNA products thereof can be administered to the host comprising the cells using any convenient protocol, where the protocol employed is typically a nucleic acid administration protocol, where a number of different such protocols are known in the art. The following discussion provides a review of representative nucleic acid administration protocols that may be employed. The nucleic acids may be introduced into tissues or host cells by any number of routes, including microinjection, or fusion of vesicles. Jet injection may also be used for intra-muscular administration, as described by Furth et al. (1992), Anal Biochem 205:365-368. The nucleic acids may be coated onto gold microparticles, and delivered intradermally by a particle bombardment device, or “gene gun” as described in the literature (see, for example, Tang et al. (1992), Nature 356:152-154), where gold microprojectiles are coated with the DNA, then bombarded into skin cells.

For example, the shRNA encoding molecules or shRNA products thereof can be fed directly to, injected into, the host organism containing the target gene. The agent may be directly introduced into the cell (i.e., intracellularly); or introduced extracellularly into a cavity, interstitial space, into the circulation of an organism, introduced orally, etc. Methods for oral introduction include direct mixing of RNA with food of the organism. Physical methods of introducing nucleic acids include injection directly into the cell or extracellular injection into the organism of an RNA solution.

In certain embodiments, a hydrodynamic nucleic acid administration protocol is employed. Where the agent is a ribonucleic acid, the hydrodynamic ribonucleic acid administration protocol described in detail below is of particular interest. Where the agent is a deoxyribonucleic acid, the hydrodynamic deoxyribonucleic acid administration protocols described in Chang et al., J. Virol. (2001) 75:3469-3473; Liu et al., Gene Ther. (1999) 6:1258-1266; Wolff et al., Science (1990) 247: 1465-1468; Zhang et al., Hum. Gene Ther. (1999) 10:1735-1737: and Zhang et al., Gene Ther. (1999) 7:1344-1349; are of interest.

Additional nucleic acid delivery protocols of interest include, but are not limited to: those described in U.S. Patents of interest include U.S. Pat. Nos. 5,985,847 and 5,922,687 (the disclosures of which are herein incorporated by reference); WO/11092; Acsadi et al., New Biol. (1991) 3:71-81; Hickman et al., Hum. Gen. Ther. (1994) 5:1477-1483; and Wolff et al., Science (1990) 247: 1465-1468; etc. See e.g., the viral and non-viral mediated delivery protocols described above.

Depending on the nature of the shRNA encoding molecules or shRNA products thereof, the active agent(s) may be administered to the host using any convenient means capable of resulting in the desired modulation of target gene expression. Thus, the agent can be incorporated into a variety of formulations for therapeutic administration. More particularly, the agents of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols. As such, administration of the agents can be achieved in various ways, including oral, buccal, rectal, parenteral, intraperitoneal, intradermal, transdermal, intracheal, etc., administration.

In pharmaceutical dosage forms, the agents may be administered alone or in appropriate association, as well as in combination, with other pharmaceutically active compounds. The following methods and excipients are merely exemplary and are in no way limiting.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The agents can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The agents can be utilized in aerosol formulation to be administered via inhalation. The compounds of the present invention can be formulated into pressurized acceptable propellants such as dichlorodifluoromethane, propane, nitrogen and the like.

Furthermore, the agents can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Those of skill in the art will readily appreciate that dose levels can vary as a function of the specific compound, the nature of the delivery vehicle, and the like. Preferred dosages for a given compound are readily determinable by those of skill in the art by a variety of means.

### Libraries

Also provided by the subject methods are complex libraries of hRNA, e.g., shRNA, expression modules, as described above. The complexity of the subject libraries (in terms of numbers of distinct shRNA expression modules) can be 1×10² or more, 1×10³ or more, 1×10⁴ or more, 1×10⁵ or more, 1×10⁶ or more, where the complexity of the product library is primarily a factor of the complexity of the input nucleic acid. A feature of the subject libraries is that the complexity and bias of the libraries is determined by the input nucleic acid. As indicated above, the input nucleic acid may be genomic DNA, a cDNA library (which may or may not be normalized), etc., such that in certain embodiments the product library may span an entire genome. Because of the nature of the subject methods, the library may include shRNA expression modules that produce shRNAs directed to both known and unknown genes, since knowledge of a gene is not required by the subject methods to produce a shRNA to that gene. Another feature of certain embodiments of the subject libraries is that they include a high percentage of expression modules that encode an shRNA molecule of appropriate size, as described above, where the number percent of such modules may be as high as 85% or higher, e.g., 90%, 95%, etc. or higher. In certain embodiments, the libraries include aproximately equal numbers of expression modules that encode the desired shRNA molecules in the sense orientation, while the remainder of the modules encode their shRNA molecules in the antisense orientiation, where the ratio of sense to antisense orientations in the product libraries may range from about 30/70 to about 70/30, such as from about 40/60 to about 60/40, including from about 45/55 to about 55/45, e.g., about 50/50.

### Systems

Also provided are systems for practicing one or more of the above-described methods. In certain embodiments, the systems are systems for producing the shRNA encoding constructs or expression modules that can be used to produce shRNA products, as described above. Such systems typically include a linker nucleic acids, e.g., pro-3′ nucleic acid, a ligase, and converting reagents, as described above. Depending on the particular protocol to be employed, the system may further include fragmentation elements, e.g., an enzyme mixture for fragmenting an initial target nucleic acid; size modification enzymes, e.g., for size modifying the a hairpin intermediate; one or more vectors; host cells; etc. In certain embodiments, the systems are systems for producing a shRNA molecule, as described above. In such embodiments, the systems include a shRNA encoding construct or expression module, e.g., present on a vector, as described above, and any other reagents desirable for transcribing the sense and antisense strands from the vector to produce the desired shRNA product, where representative reagents include host cells, factors, etc.

### Kits

Also provided are reagents and kits thereof for practicing one or more of the above-described methods. The subject reagents and kits thereof may vary greatly. In certain embodiments, the kits include at least a linker nucleic acid, e.g., a pro-3′ nucleic acid. The subject kits may further include one or more of: a ligase, converting reagents, fragmentation elements, e.g., an enzyme mixture for fragmenting an initial target nucleic acid, size modification enzymes, e.g., for size modifying a hairpin intermediate, one or more vectors, host cells, etc., as described above. In certain embodiments, the kits at least include the subject shRNA encoding constructs, and any other reagents desirable for transcribing the sense and antisense strands from the vector to produce the desired shRNA product, where representative reagents include host cells, factors, etc.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

The following examples are offered by way of illustration and not by way of limitation.

### EXPERIMENTAL

### I. Materials and Methods

### A. Amplification of genes used for REGS

The open reading frames for the glucocorticoid receptor (GR), eGFP, MyoD, and Oct-3/4 were generated by PCR amplification using the following primers:

glucocorticoid receptor (2268 bp) GR
forward:

| | |
|---|---|
| 5′ ATGGACTCCAAAGAATCC 3′; | (SEQ ID NO:01) |
| and | |
| reverse: | |
| GAATTCAATACTCATGGA 3′; | (SEQ ID NO:02) |
| eGFP (721 bp) eGFP | |
| forward: | |
| 5′ AACCATGGTGAGCAAGGGCGA 3′; | (SEQ ID NO:03) |
| and | |
| reverse: | |
| 5′ CTTGTACAGCTCGTCCATGCC 3′; | (SEQ ID NO:04) |
| MyoD (960 bp): | |
| forward: | |
| 5′ATGGAGCTTCTATCGCCGCC3′; | (SEQ ID NO:05) |
| and | |
| reverse: | |
| 5′ TCTCTCAAAGCACCTGATAA3′; | (SEQ ID NO:06) |
| OCT-3/4 (1324 bp): | |
| forward: | |
| 5′GTGAGCCGTCTTTCCACCA3′; | (SEQ ID NO:07) |
| and | |
| reverse: | |
| 5′ACTGTGTGTCCAGTCTTT3′. | (SEQ ID NO:08) |

### The PCR cycle consisted of 30 cycles at 94° C./1 min., 60° C./1 min., and 72° C./1 min. for all genes except for GR which was cycled at 94° C./1 min., 53° C./1 min. and 72° C./3 min. for 30 cycles.

### B. VREGS Generation

A 425 bp stuffer sequence derived from the Oct-3/4 open reading frame was created using a 5′ primer (REGS STUFF A) containing a Bglll site [5′GGGAAGATCT(Bglll)GCCGACAACAATGAGAACCTT3′] (SEQ ID NO:09) and a 3′primer (REGS STUFF B) containing Hindlll and Bbsl_sites [5′GCCCAAGCTT(Hindlll)TCCAAAAAAAGTCTTC (Bbsl)CAGAGCAGTGACGGGMCAG3′] (SEQ ID NO: 10). The primers were used to amplify the stuffer sequence from cDNA derived from embryonic stem cells. The product was cloned into the Bglll/Hindlll site of pSuper retroviral vector (Oligoengine) thus creating vREGS. To prepare the vector for siRNA insertion, vREGS was digested with Bglll/Bbsl. The Bbsl site cuts 6 nucleotides away leaving the 4 nucleotide 5′ TTTT 3′ overhang. T4 DNA polymerase was used to fill in the overhangs left by Bbsl allowing the formation of a blunt end.

### C. The REGS Process (See FIG. 1)

Step 1, 5 μg of each gene was digested with Hinpl, BsaHl, Acil, Hpall, HpyCHIV, and Taq∝l (New England Biolabs) and purified using Qiaex II beads (Qiagen).

Step 2. 3 μg of the digested gene fragments were ligated to 1.5 μg (2:1 ratio) of the 3′ loop (5′CGTTGGATCCCGGTTCAAGAGACCGGGATCCAA 3′) (SEQ ID NO:11) for 1 hour and heat inactivated at 65° C. for 10 minutes. All loop oligonucleotides were ordered PAGE purified from Integrated DNA Technologies. The reaction was diluted 3-fold into Mmel buffer including SAM and the Mmel enzyme (NEB) for 1 hour. The reaction was run on a 20% TBE Novex gel (Invitrogen) and the ˜34 bp (gene fragment+3′loop) was excised, fragmented into small pieces, and placed in 0.5 M salt for 3-5 hours at 50° C. Qiaex II beads (Qiagen) were used to purify the DNA from the salt solution according to manufacturer's instructions.

Step 3, 1 μg of the purified band was ligated to 500 ng of 5′loop(5′GGAGAGACTCACTGGCCGTCGTTTTACCAGTGAAGATCTCCNN3′) (SEQ ID NO:12) (2:1 ratio) for 1.5 hours run on a 10% TBE Novex gel and the ˜60 bp band was gel purified.

Step 4, Rolling circle amplification (RCA) was performed using the TempliPhi 100 amplification kit according to manufacturer's protocol (Amersham Biosciences) except primers RCA1 (5′ACTGGTM3′) (SEQ ID NO:13) and RCA2 (5′GCCGTCGT3′) (SEQ ID NO:14) specific to the 5′ loop were used. The RCA reaction was incubated at 30° C. for 12 hours and heat inactivated at 65° C. for 10 minutes.

Step 5, RCA products were diluted 1:2 into buffer 2 (NEB) containing Bglll and Mlyl. The desired fragment (82 bp) was isolated from a 10% TBE gel. 30 ng of the Bglll/Mlyl fragment was ligated to 90 ng of vREGS (1:3 ratio) and transformed into Stbl2 bacterial competent cells (Invitrogen). Resulting bacterial colonies were scraped and the siRNA constructs isolated using a mini prep kit (Qiagen).

Step 6, The plasmids were then digested with BamHl and self-ligated to produce the final siRNA constructs. Individual colonies were picked and plasmids isolated. The constructs were digested with BamHl prior to sequencing in order to prevent the formation of secondary structure caused by the palindromic nature of the cloned inserts.

### D. REGS Library

The double stranded cDNA from a mouse embryonic retroviral library (Clontech) was isolated from the vector sequences by digesting with Sfil (New England Biolabs) and gel purified. The protocol is the same as used for the other genes except for the noted changes. 5 μg of double stranded cDNA were used as starting material for the first ligation and all loop amounts were scaled accordingly. Step 4, Twenty RCA reactions were performed at 30° C. for 2 hours. The colonies resulting from completion of Step 5 were counted to determine the complexity of the library. Dilutions that ranged from 0.45 ng, 0.9 ng, 45 ng, and 9 ng of vector DNA were used to determine the number of colonies yielded per microgram of vector DNA.

### E. Cell Culture

Primary myoblasts were isolated from adult FVBNJ mice and grown in DMEM with 20% FCS and bFGF as previously described (Tiscornia et al., Proc. Nat'l Acad. Sci. USA (2003) 100: 1844-8). Differentiation assays were done by placing myoblasts in DMEM with 5% horse serum for two days. Embryonic stem cells, line D3, were obtained from the ATCC and grown in Knockout DMEM (GIBCO), 15% knockout serum (GIBCO), and Lif (ESGRO from Chemicon).

### F. Stable Cell Line Production

Ecotropic phoenix cells (gift from Garry Nolan) were transfected with 1.6 μg of each REGS pSuper siRNA constructs. Transfections were done in 12 well plates using Lipofectamine 2000 (Invitrogen) according to manufacturers instructions. Viral supernatants were collected 48 hours post transfection and polybrene added (5 μg/ml). These supernatants were placed on target cells and centrifuged for 30 minutes at 2,000×g. Cells were infected four times and selected with puromycin (1 μg/ml) one day after the last infection.

### G. Generation of eGFP Expressing Primary Myoblasts

eGFP was cloned into the MFG retroviral vector and transduced into adult FVBNJ primary myoblasts. Individual cells were sorted and cloned using the Facstar cell sorter (Becton Dickinson). One clone was subsequently used for all GFP experiments.

### F. Western Blot Analysis

Cells were trypsinized and -pelleted through centrifugation. Cells were resuspended and lysed in buffer containing 1% Nonidet (NP-40), 150 mM NaCl, 50 mM Tris pH 8.0, 1 mM EDTA, 0.1% SDS, 0.5% Na-Deoxycolate, and a protease inhibitor cocktail (Roche). Samples were quantitated using BioRad's protein assay according to manufacturer's instructions. 1 μg of total protein was loaded for all samples in the analysis for eGFP and ∝-Tubulin expression. 5 μg of total protein was loaded for expression analysis of MyoD. Samples were run on NuPAGE 4-12% Bis-Tris gradient gels (Invitrogen) and transferred to lmmobilon-P (Millipore) for immunoblotting. Polyclonal rabbit anti-GFP antibody (Molecular Probes, A-11122) was used at a dilution of 1:6000, mouse anti-∝-tubulin antibody (Sigma, T5168) and mouse anti-MyoD antibody (PharMingen, 554130) were used at 1:1000. HRP conjugated, goat anti-mouse (Zymed Laboratories, 81-6520) and goat anti-rabbit (Zymed Laboratories, 81-6120) secondary antibodies were used at a dilution of 1:5000. Blots were detected using ECL (Amersham Biosciences) according to manufacturer's protocol. Signals were quantitated using a Lumi-Imager (Mannheim Boehringer). The densitometric data obtained from the eGFP or MyoD band was normalized to ∝-Tubulin. The densitometric data from the control was set at 100% and all other data were represented as a percentage of the control value.

### G. RNA Isolation and Semi-Quantitative RT-PCR

Total RNA was extracted from embryonic stem cells using the RNeasy mini kit (Qiagen. 1 μg of total RNA was reverse transcribed using the 1^{st} Strand cDNA Synthesis Kit for RT-PCR (Roche). 1 μl of cDNA was used for amplification using the Titanium Taq PCR kit from Clontech. The PCR cycle for all reactions consisted of 94° C./1 min., 60° C./1 min. and 72° C./1 min. with number of cycles dependent on each gene. The primer sequences for Oct-3/4, UTF1, ESG-1, and H19 were:

| | |
|---|---|
| Oct-3/4 | |
| forward | |
| 5′ GCCGACAACAATGAGAACCTT 3′, | (SEQ ID NO:15) |
| reverse | |
| 5′ CAGAGCAGTGACGGGAACAG 3′ | (SEQ ID NO:16) |
| UTF1 | |
| forward | |
| 5′ GTCCCTCTCCGCGTTAGCA 3′, | (SEQ ID NO:17) |
| reverse | |
| 5′ AGCTTTATTGGCGCAAGTCCC 3′, | (SEQ ID NO:18) |
| ESG-1 | |
| forward | |
| 5′ ACCCTCGTGACCCGTAAAGAT 3′, | (SEQ ID NO:19) |
| reverse | |
| 5′ TCGATACACTGGCCTAGCTCC 3′ | (SEQ ID NO:20) |
| H19 | |
| forward | |
| 5′ TGTATGCCCTAACCGCTCAG 3′, | (SEQ ID NO:21) |
| reverse | |
| 5′AACAGACGGCTTCTACGACAA 3′. | (SEQ ID NO:22) |

Mouse β-actin primers were purchased from Stratagene (302110). Semi-quantitative RT-PCR on Oct-3/4 was performed by running for 21, 24 and 27 cycles, β-Actin for 19, 21, and 23 cycles, UTF1 for 25 and 27 cycles, ESG1 for 21 and 23 cycles and H19 for 21 and 24 cycles. PCR products were visualized on 1% agarose gels stained with ethidium bromide.

### H. Alkaline Phosphatase Staining and Immunofluorescence

Embryonic stem cells were fixed and stained using the Alkaline Phosphatase staining kit (Sigma, 85L-2) according to manufacturer's instructions. For immunofluorescence, cells were fixed in 4% paraformaldehyde for 5 minutes and blocked in buffer containing 2.5% normal goat serum, 0.3% triton×100, and 2% BSA for 30 minutes. Mouse anti-∝-sarcomeric actin (Sigma, A-2172) and rabbit anti-GFP (Molecular Probes, A-11122) were used at 1:200 and 1:2500 respectively. Secondary antibodies were Texas Red conjugated goat anti-mouse IgM (Jackson, 115-075-075) (1:1000), and Alexa 488 conjugated goat anti-rabbit (Molecular Probes, A-11034) (1:1000).

### II. Results

### A. REGS Process

The procedure for generating siRNAs in quantity from double stranded cDNAs is outlined and described briefly in FIG. 1. Features of the Restriction Enzyme Generated siRNA (REGS) procedure and the rationale behind each step are described in detail below. Although REGS was performed on 4 genes, GFP, Oct-3/4, MyoD, and the glucocorticoid receptor (GR), the process will only be described for GR and functional data of the siRNAs generated are provided for the other three genes.

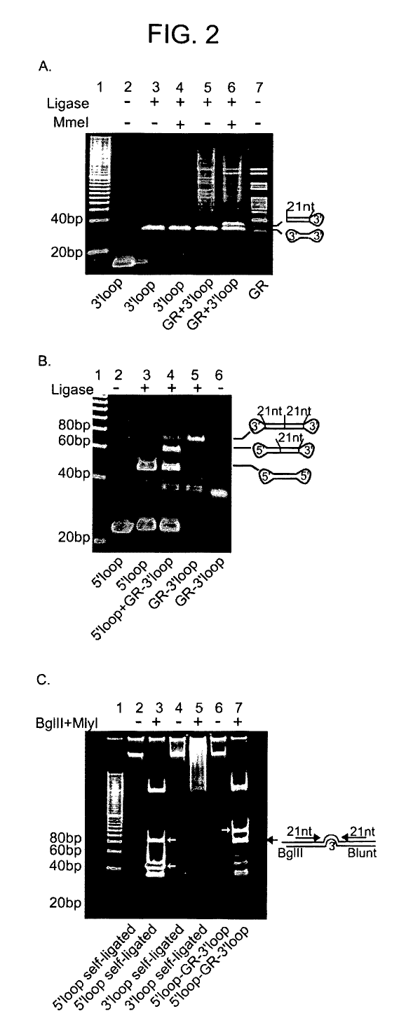

First, restriction enzymes were selected that would yield a large number of fragments per gene in the genome and generate identical 2 bp overhangs to facilitate future ligation of these fragments (Step 1, FIG. 1). A survey of the commercially available restriction enzymes revealed an abundance of enzymes that not only cut frequently (˜4 bp recognition site) in the mouse genome but also leave a 5′ CG overhang (Hinpl, BsaHl, Acil, Hpall, HpyCHIV, and Taq∝l). A mixture of these enzymes would be expected to cut a random sequence once every 25 bp, however a computer analysis of 10 randomly selected. mouse genes revealed that these enzymes cut coding regions an average of once every 80 bp, possibly due to the CG requirement of the center base pairs. GR was digested using the restriction enzyme cocktail (FIG. 2a, lane 7).

Second, the sense and antisense strands of the gene fragments were linked by ligation to a 3′ hairpin loop. The purpose of the hairpin loop linking the strands is to allow the complementary strand to be synthesized. This hairpin DNA oligonucleotide, the 3′ loop, contains the requisite 5′CG overhang to allow ligation (Step 2, FIG. 1). As a result, once the complementary strand is synthesized, the sequence forms a palindromic structure that encodes a functional siRNA molecule.

Only fragments of the appropriate size encode functional siRNAs. The fragments ligated to the 3′ loop differed markedly in size (FIG. 2a, lane 5). Most fragments exceeded 29 bp rendering them incompatible with siRNA expression because double stranded RNA longer than 29 bp elicits an interferon response in mammalian cells. Using only these methods, 1, 4, 2, and 15 sequences of a size compatible with the generation of siRNAs would be obtained from GR, GFP, Oct-3/4 and MyoD respectively. To generate fragments of a suitable size and to increase the number of clonable fragments, a partial restriction enzyme site (Mmel) was engineered adjacent to the ligation site of the 3′ loop. Upon ligation of this loop to the gene fragments, the complete enzyme recognition site (5′ TCCPuAC 3′) for Mmel was formed. Mmel cuts a distance of 20 bp, 3′ from its recognition sequence. In this manner all fragments greater than 21 nt will generate 2 clonable siRNA sequences because the 3′loop can ligate to either terminus and the ensuing Mmel digestion generates two products of the appropriate size. The last C of the Mmel site overlaps the first nucleotide of the gene sequence because the initial fragments generated end in a CG overhang. This base plus the 20 bp fragment generates 21 bp of gene specific sequence. Digestion of the ligation product with Mmel generates a band at 34 bp which includes 21 bp of gene specific sequence ligated to the 13 bp 3′ loop, (FIG. 2a, lane 6), terminating in a 3′2 bp overhang of random sequence (NN). In order to generate a DNA sequence that would encode a functional siRNA, the Mmel digested hairpin loop structure had to be linearized and the complementary strand synthesized. To generate priming sites that would allow the synthesis of the complementary strand an adapter, 5′loop, was ligated to the 2 bp overhang left by the Mmel digestion (Step 3, FIG. 1). The 5′loop consists of a 43 nt hairpin oligonucleotide predicted to form a 15 bp stem loop ending in a 3′ NN extension that is compatible with the overhangs left by the Mmel digestion. After PAGE purification, the 3′ loop +21 bp gene sequence was ligated to the 5′ loop. The 5′ loop ligates to itself (FIG. 2b, lane 3), but also ligates efficiently to the 3′loop+21 bp fragment as is evident from the appearance of the 60 bp band (FIG. 2b, lane 4) (Step 4, FIG. 1).

The stability of the central double stranded region in the ligation product impedes efficient synthesis of the complementary strand and amplification by conventional PCR. Thus, a strand displacing enzyme, Phi 29 DNA polymerase, was chosen to synthesize the complementary strand and amplify the ligation product by rolling circle amplification (RCA). The 5′loop-GR fragment-3′loop was PAGE purified and amplified using isothermal rolling circle amplification (RCA) for 12 hours at 300° C. Primer RCA1, specific to the 5′ loop was added to the circular structure to prime Phi 29 which disrupts the hairpin structure and synthesizes the complementary strand. The enzyme continues to replicate the DNA around the dumbbell, displacing the newly synthesized strand and with each successive completion of the circle amplifies the ligation product, thus generating a long ssDNA concatemer. The RCA2 primer, also specific to the 5′loop, was included in the reaction to prime the complementary strand and create a dsDNA concatemer.

To isolate the final DNA products with the appropriate structure, the concatemers resulting from the RCA reaction were digested with Bglll and Mlyl (FIG. 1 Step 5). Digestion of the concatamerized RCA product with these enzymes generates an 82 bp fragment that encodes the clonable siRNA sequence (FIG. 2c, lane 7), and a 38 bp fragment containing the 5′ loop. The band slightly above at 109 bp is the result of incomplete digestion with Mlyl. The 5′loop ligated to itself (self-ligated) and then amplified by RCA yields the expected band at 38 bp, in addition to partial digestion products at 44 and 80 bp following incubation with the restriction enzyme Mlyl (FIG. 2c, lane 3).

The REGS process was designed to generate products that ultimately contain no extraneous sequences that could hinder siRNA expression. To this end, the Mlyl site was incorporated 5 bp upstream of the last siRNA nucleotide. Digestion with Mlyl generates a blunt end directly following the siRNA sequence. To allow ligation of the Bglll/Mlyl digested product, the original pSuper retroviral vector (Brummelkamp, Science (2002) 296: 550-3) was modified so that the 3′ cloning site could be blunt ended immediately preceding the RNA polymerase lll termination site TTTTTGGAA; this vector was designated vREGS. As a result, insertion of the digested 82 bp REGS products downstream of the H1 RNA polymerase promoter into the Bglll blunt ended vector sites culminated the desired product devoid of extraneous sequences.

The E. coli colonies obtained from this cloning reaction were scraped, pooled and plasmid DNA isolated. However, this product still included excess 3′loop. The 3′ loop was intentionally made longer than useful for siRNA production to ensure efficient self annealing and ligation to the gene fragments by T4 DNA ligase (FIG. 1, Step 2). A BamHl site had been previously included in the 3′ loop that was replicated during RCA to form opposing BamHl sites that bordered the excess sequence to allow its removal (Step 6, FIG. 1). Following digestion with BamHl, re-ligation of the plasmid pool resulted in expression-ready siRNA vectors.

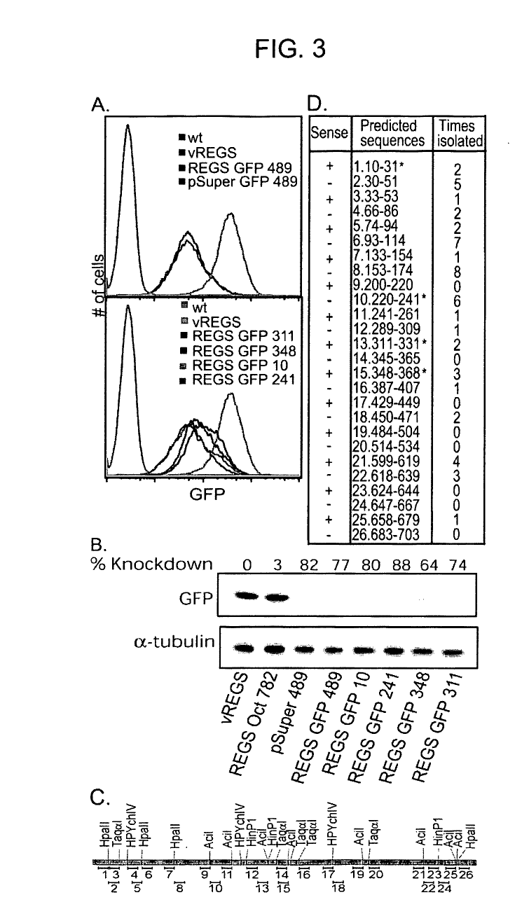

The only difference between the products of REGS and conventionally created siRNAs is the loop structure that connects the sense and antisense sequences. To test whether the inclusion of the vREGS-specific loop (Transcribed, FIG. 1) affected siRNA function, we compared the previously published pSuper loop with the vREGS loop. Four 19 nt siRNAs to GFP were generated with the pSuper loop and cloned into pSuper Retro by traditional oligonucleotide synthesis. The sequence corresponding to nt 489-597 had been previously found to mediate efficient silencing (data not shown). This GFP siRNA sequence was then cloned using the vREGS loop. Both constructs were transfected into packaging cells and supernatants were used to infect primary myoblasts previously engineered to constitutively express GFP. The pSuper GFP 489 and vREGS GFP 489 constructs both showed a 10-fold decrease in GFP fluorescence when analyzed by flow cytometry (FIG. 3a, upper panel). Western blot analysis showed an 82 and 77% silencing of GFP by pSuper GFP 489 and REGS GFP 489 respectively (FIG. 3b). Thus, the knockdown of GFP was essentially the same irrespective of loop structure.

To determine the representation of the possible products from a single gene, we performed the REGS procedure on GFP and analyzed 52 resulting clones. FIG. 3c shows the possible siRNA sequences generated from GFP. Of the 52 sequenced plasmids, we obtained 18 unique siRNA retroviral constructs for GFP of a total of 26 possible (FIG. 3d).

REGS facilitates both the cloning of sense and antisense orientation with equal probability and, as expected, half of the 18 unique constructs were cloned with the 21 mer sense-strand 5′ to the loop (sense orientation) (FIG. 3d). Four of the nine sense constructs showed knockdown of GFP when transduced into primary myoblasts constitutively expressing GFP, whereas none of the antisense constructs were effective, consistent with reports by Czauderna et al., Nucleic Acids Res. (2003) 31: 670-82. siRNAs 10-31, and 241-261 exhibited nearly a 10-fold knockdown of GFP expression by flow cytometry, whereas GFP 311-331 and 348-368 showed approximately an 8-fold knockdown (FIG. 3a, lower panel). Western blot analysis (FIG. 3b) was consistent with the flow cytometry data showing 80% knockdown for GFP 10-31, 88% for GFP 241-261, 64% for GFP 348-368, and 74% for 311-331.

### B. Knockdown of Endogenous Genes by REGS Vectors

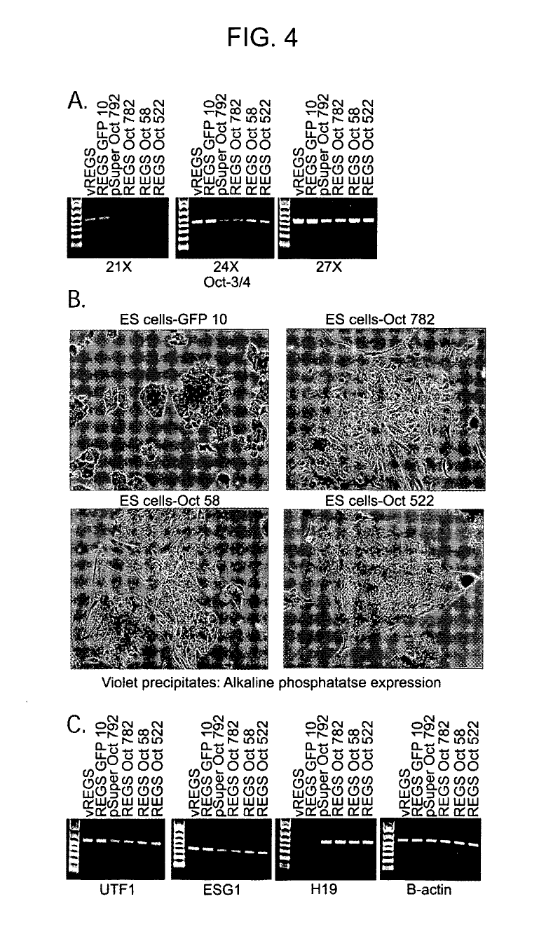

We tested the efficacy of siRNA molecules generated by REGS to silence the Oct-3/4 gene in embryonic stem(ES) cells. (Oct-3/4 is a transcription factor that is essential for the self renewal of ES cells). Reduction in Oct-3/4 expression results in the differentiation of ES cells to trophoblasts, providing a phenotypic assay for loss of Oct-3/4 gene expression. Using REGS, we obtained 6 sense and 5 antisense constructs. Three of the sense strand sequences, 58-78, 522-541, and 782-803 showed knockdown of Oct-3/4 (FIG. 4a). Oct 782 showed the greatest suppression. The degree of Oct 782 suppression was on a par with Oct 792-811, which had previously been constructed in pSuper Retro by traditional methods and shown to mediate silencing (data not shown). Oct 782 and 792 both showed greater than 8-fold reduction of Oct-3/4 message by semi-quantitative RT-PCR, while Oct 58 and 522 showed slightly less (FIG. 4a, center panel). All three constructs caused the differentiation of ES cells to trophoblasts evidenced by large, flattened cell morphologies, and a subsequent loss of alkaline phosphatase staining (FIG. 4b). This change in phenotype was accompanied by the downregulation of other genes associated with ES cells, UTF1 and ESG-1, which are both highly expressed in undifferentiated stem cells while H19, a marker for ES cell differentiation, was highly upregulated (FIG. 4c)

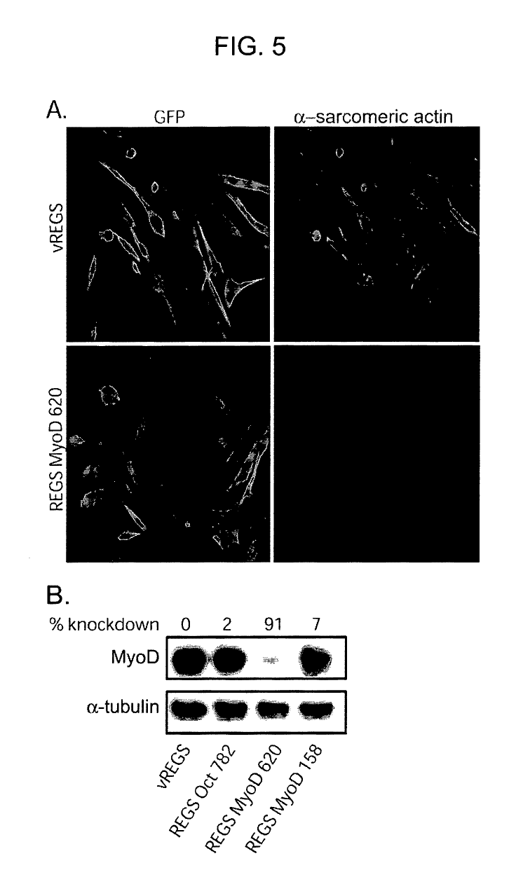

Another example of REGS-mediated silencing of an endogenous gene is provided by MyoD. MyoD is a basic helix loop helix transcription factor that is essential for the differentiation of myoblasts to myotubes. Primary myoblasts that constitutively expressed GFP were transduced with 6 sense siRNA constructs generated from MyoD using REGS. These cultures were differentiated in low mitogen medium for 2 days and then assayed for their ability to form myotubes and express differentiation specific genes. The siRNA corresponding to MyoD 620-640 was found to block differentiation completely as shown by the absence of myotube formation and alpha-sarcomeric actin staining (FIG. 5a). Western blot analysis of these cells cultured in growth medium showed a 91% knockdown of MyoD expression by REGS MyoD 620, whereas another sense-strand construct, REGS MyoD 158 showed little effect (FIG. 5b). These results show that the REGS generated siRNAs are functional as they significantly inhibit gene expression and alter cell fate.

### C. Construction of a REGS Library

The advantage of the REGS system presented here is the ability not only to produce large numbers of unique siRNA constructs simultaneously per gene, but also to generate sufficient numbers to yield an siRNA library that spans the entire genome. To test this possibility, we obtained a murine embryonic retroviral library. The inserts were excised from the parental plasmid by restriction digest and gel purified. The rest of the cloning procedures were essentially identical to those described in FIGS. 1 and 2 for REGS, except Step 4 in which twenty RCA reactions were carried out for 2 hours, instead of a single reaction for 12 hours. The number of reactions was increased and length of reaction time decreased to enhance the complexity of the library. The number of independent colonies obtained from the first transformation (Step 5) was assessed to determine the complexity of the siRNA library. Dilutions ranging from 0.45 ng, 0.9 ng, 4.5 ng, and 9 ng of vector DNA were used to establish the number of colonies obtained per microgram of vector DNA. From this value, we calculated the library complexity to be 415,000 independent siRNA constructs/ug of vector DNA.

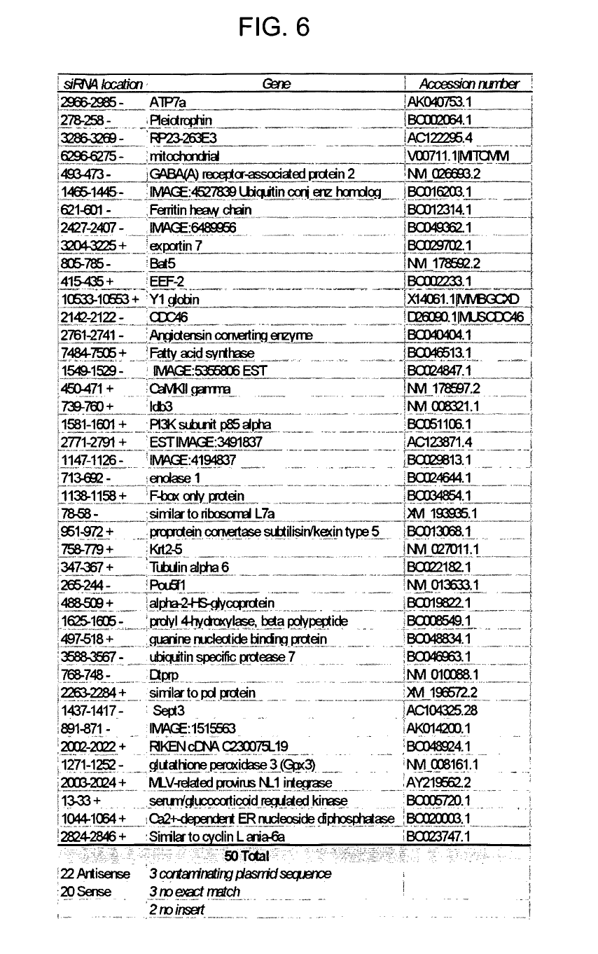

50 independent constructs were isolated and sequenced from the library. Of these, 48 constructs contained inserts with the appropriate structures and all were unique (FIG. 6). 42 of these clones had sequences identical to GenBank entries (FIG. 6) with approximately one-half cloned in the sense orientation. Three clones had no exact match in the mouse genome and another three had sequences obtained from the parental plasmid. Only 2 constructs were found that contained no inserts. These results show that REGS can be used to generate a high complexity library(>4×105) in 4 days with greater than 96% of the clones containing double stranded DNA encoding siRNA inserts of the appropriate size.

### III. Discussion

Although several groups have recently developed vectors encoding short hairpin RNA molecules that mediate specific gene silencing, the utility of these vectors is only beginning to be realized and their versatility exploited. A major drawback shared by all existing approaches to create siRNA vectors is the expense and inefficiency associated with their construction, generally limiting the application of this technology to one or only a few genes. In this report, we describe a facile method, REGS, for generating a multitude of siRNA constructs that target either an individual gene or pool of cDNAs. We show that the REGS generated vectors are identical in form and function to traditionally created vectors by directly comparing the same siRNA sequence targeting GFP using the vREGS or pSuper loop.

The REGS vectors were further tested in their ability to silence endogenous genes such as Oct-3/4, and MyoD. Three siRNAs generated from Oct-3/4 activated differentiation in ES cells resulting in trophoblast formation and loss of alkaline phosphatase expression. An siRNA generated from MyoD blocked myoblast differentiation demonstrated by an absence of myotube formation and ∝-sarcomeric actin expression. Different sequences isolated from GFP and Oct-3/4 genes mediated gene silencing to significantly different degrees, from 64 to 88%. Thus, the most efficient siRNAs generated by REGS reduced gene expression to approximately 10% of wild type levels. Because REGS generates a large number of distinct sequences, suppression of gene expression to different extents can be achieved using this siRNA based technology and readily extended to studying haplo-insufficiency and other effects of gene dosage.

To date, it remains unclear why some siRNA sequences function better than others. Most investigators report that 25% of siRNA constructs are capable of suppressing the gene to which they are targeted. Our frequencies are in good agreement with those findings as, on average, 1 of 3 sense strand constructs silenced the three genes tested, GFP (4 of 9 constructs), Oct-3/4 (3 of 6 constructs), and MyoD (1 of 6 constructs). Thus an advantage of REGS is that due to the large number of unique siRNAs that can be readily generated, the isolation of functional siRNA vectors to any given gene is highly likely.

Efforts are underway to develop siRNA vectors against every gene in the human genome. The labor intensive cloning process associated with generating at least four constructs for each of the 40,000 genes in the genome using current methods is generally overwhelming. By contrast, using REGS, we were able to generate a siRNA library including approximately 415,000 inserts using a cloning process that requires only 3-4 days. For high-throughput screening, individual clones from these libraries could be isolated and sequenced to generate arrayed libraries or the library could be screened as a whole in a manner similar to that used for cDNA library screening. Such libraries could easily be generated for any given organism, tissue, or cell type. In addition, siRNA libraries generated from cDNA populations have the advantage of isolating unknown targets or differentially spliced and disease related transcripts.

As the REGS generated library is the first of its kind, several aspects bear noting. The restriction enzymes used by REGS generate more fragments from longer DNA sequences, whereas the reverse transcriptase used to generate cDNA libraries is more efficient with smaller genes. Consequently, the REGS generated RNA libraries are biased toward larger genes in contrast with conventional cDNA libraries. In addition, by using restriction enzymes that recognize different sets of 4 base pair sequences at the initial step of this process, diverse sets of fragments can be generated so that the gene(s) of interest can be entirely encompassed. Furthermore, all of the inserts are the same size, preferential amplification of certain sequences within the library is not likely to occur as the library is expanded.

Although less than two years have passed since the first reports of DNA-based RNAi, an abundance of different RNAi applications and distinct vector-based RNAi systems have been published. For example, there are now a variety of reports using viral vectors (lentiviral and retroviral), inducible systems, and even the generation of loss of function transgenic mice using RNAi. In addition, improvements are constantly being made to the vectors themselves. The simplicity of the REGS technology described here allows both the generation of numerous gene-specific siRNAs that can be easily interchanged between the different vector types as well as the generation of complex RNAi libraries from any eukaryotic organism.

It is evident from the above results and discussion that the subject invention provides improved methods of producing siRNAs, as well as improved methods of using the produced siRNAs in various applications, including high throughput loss of function applications. A particular advantage of the subject invention is the ability to use the methods to rapidly and efficiently (as well as inexpensively) produce highly complex libraries from a variety of different input nucleic acids, including genomic libraries, cDNA libraries, etc., where the libraries can include shRNA encoding molecules directed to both known and unknown genes. As such, the subject invention makes the low cost rapid determination of gene function possible. Accordingly, the present invention represents a significant contribution to the art.

All publications and patents cited in this specification are herein incorporated by reference as if each individual publication or patent were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1. A method of producing an hRNA expression module for a specific target nucleic acid, said method comprising:
(a) ligating a linker nucleic acid to an initial dsDNA that corresponds to said shRNA to produce a single-stranded intermediate nucleic acid that comprises a linker domain flanked by intra-complementary domains; and
(b) converting said intermediate nucleic acid to a linear dsDNA that includes at least one copy of said shRNA expression module, where said expression module comprises a linker domain flanked by hRNA coding domains.

2. The method according to claim 1, wherein said method further comprises producing said initial dsDNA from said specific target nucleic acid.

3. The method according to claim 2, wherein said initial dsDNA is produced by fragmenting said target nucleic acid.

4. The method according to claim 3, wherein said target nucleic acid is enzymatically fragmented.

5. The method according to claim 4, wherein said hRNA expression module is an shRNA expression module.

6. The method according to claim 4, wherein said two or more restriction endonucleases are selected to produce an enzyme combination that cleaves said target nucleic acid into fragments of a predetermined size.

7. The method according to claim 1, wherein said method further comprises size modifying said intermediate nucleic acid.

8. The method according to claim 7, wherein said intermediate nucleic acid is enzymatically size modified.

9. The method according to claim 1, wherein said converting step does not include an amplification step.

10. The method according to claim 1, wherein said converting step includes an amplification step.

11. The method according to claim 10, wherein said amplification comprises PCR.

12. The method according to claim 10, wherein said amplification comprises rolling circle amplification.

13. A method of producing a shRNA specific for a target nucleic acid molecule, said method comprising: producing an expression module for said shRNA according to the method of claim 1; and transcribing said expression module to produce said shRNA.

14. The method according to claim 13, wherein said method is in vitro.

15. The method according to claim 13, wherein said method occurs inside of a cell and said method further comprises introducing said expression module into said cell.

16. The method according to claim 13, wherein said expression module is present on a vector.

17. A single stranded nucleic acid comprising complementary domains separated by a linker domain, wherein said complementary domains hybridize to each other to produce a hairpin structure having a double-stranded stem domain and single stranded loop domain, wherein said double-stranded stem domain comprises a restriction endonuclease site.

18. The nucleic acid according to claim 17, wherein said restriction endonuclease site is a substrate for an endonuclease that cleaves a nucleic acid at a cleavage site that is a defined distance from said site.

19. The nucleic acid according to claim 18, wherein said defined distance is from about 10 to about 40 bp.

20. The nucleic acid according to claim 18, wherein said double stranded stem domain further comprises at least one additional restriction endonuclease site.

21. A single-stranded intermediate nucleic acid that comprises a linker domain flanked by intra-complementary domains, wherein said intermediate nucleic acid comprises a nucleic acid according to claim 17.

22. A closed circular single-stranded DNA molecule comprising a nucleic acid according to claim 21.

23. A linear dsDNA that comprises at least one pro-shRNA expression module made up of a linker domain flanked by siRNA encoding domains, wherein said linker domain comprises two restriction endonuclease sites.

24. The linear dsDNA according to claim 23, wherein said dsDNA comprises at least two pro-shRNA expression modules.

25. The linear dsDNA according to claim 23, wherein said two restriction endonuclease sites of said linker domain are identical.

26. The linear dsDNA according to claim 23, wherein said linker domain ranges in length from about 4 to about 25 bp.

27-33. (canceled)

34. A system for producing an shRNA expression module for a specific target nucleic acid, said system comprising: a nucleic acid according to claim 17; a ligase for ligating said nucleic acid to an initial dsDNA; and converting reagents for converting an intermediate nucleic acid to a linear dsDNA that comprises at least one shRNA expression module.

35-58. (canceled)
